(19) United States
(12) Patent Application Publication
(10) Pub. No.: US 2004/0192898 A1
(43) Pub. Date: Sep. 30, 2004

(54) Anti-abeta antibodies

(76) Inventors: Audrey Yunhua Jia, Union City, CA (US); Naoya Tsurushita, Palo Alto, CA (US); Maximiliano J. Vasquez, Palo Alto, CA (US)

Correspondence Address: Thomas LaGrandeur, Eli Lilly & Company, Patent Division, PO Box 6288, Indianapolis, IN, 46206-6288, US

(21) Appl. No.: 10/487,322

(22) PCT Filed: Aug. 14, 2002
(86) PCT No.: PCT/US02/21322

Related U.S. Application Data
(60) Provisional application No. 60/313,224, filed on Aug. 17, 2001.

(51) Int. Cl. C07K 16/18

(52) U.S. Cl. 530/388.1

(57) ABSTRACT

This invention provides variant 266 antibodies that are engineered to lack an N-glycosylation site within the CDR2 of the heavy chain, pharmaceutical compositions thereof, and polynucleotide sequences, vectors, and transformed cells useful to express the variant antibodies. The variants sequester soluble Aβ peptide from human biological fluids and specifically bind an epitope contained within position 13-28 of the amyloid beta peptide Aβ with significantly greater affinity than either mouse antibody 266 or humanized 266 antibodies retaining N-glycosylation sites. The variant antibodies are useful for treatment or prevention of conditions and diseases associated with Aβ, including Alzheimer's disease, Down's syndrome, cerebral amyloid angiopathy, mild cognitive impairment, and the like.

This application claims the priority of U.S. provisional application 60/313,224, filed Aug. 17, 2001, the entire contents of which are incorporated by reference.

### BACKGROUND OF THE INVENTION

The invention relates to analogs of antibody 266 that lack an N-glycosylation site in the second complementarity determining region (CDR2) of the heavy chain. Such antibodies are useful for preventative and therapeutic treatment of conditions associated with the Aβ peptide, such as Alzheimer's disease, Down's syndrome, and cerebral amyloid angiopathy.

A number of conditions and diseases appear to be associated with neuritic and cerebrovascular plaques in the brain containing amyloid beta peptides (Aβ). Among these are both pre-clinical and clinical Alzheimer's disease, Down's syndrome, and pre-clinical and clinical cerebral amyloid angiopathy (CAA). The Aβ peptide in circulating form is composed of 39-43 amino acids (mostly 40 or 42 amino acids) resulting from the cleavage of a precursor protein, amyloid precursor protein (APP).

Methods to induce an immune response to reduce amyloid deposits are described in PCT publication WO99/27944 published 10 Jun. 1999. The description postulates that full-length aggregated Aβ peptide would be a useful immunogen. Administration of a Aβ fragment (amino acids 13-28) conjugated to sheep anti-mouse IgG caused no change in cortex amyloid burden, and only one in nine animals that received injections of the Aβ 13-28 fragment-conjugate showed any lymphoproliferation in response to Aβ₄₀. The application also indicates that antibodies that specifically bind to Aβ peptide could be used as therapeutic agents. However, this appears to be speculation since the supporting data reflect protocols that involve active immunization using, for example, Aβ₄₂.

WO 00/72880 and Bard, F., et al., Nature Med. (2.000) 6:916-919 describe significant reduction in plaque in cortex and hippocampus in a transgenic mouse model of Alzheimer's disease when treated using N-terminal fragments of Aβ peptides and antibodies that bind to them, but not when treated with the Aβ 13-28 fragment conjugated to sheep anti-mouse IgG or with an antibody against the 13-28 fragment, antibody 266. The N-terminal directed antibodies were asserted to cross the blood-brain barrier and to induce phagocytosis of amyloid plaques in in vitro studies.

WO 00/77178 describes antibodies that were designed to catalyze the hydrolysis of β amyloid, including antibodies raised against a mixture of the phenylalanine statine transition compounds Cys-Aβ₁₀₋₂₅, statine Phe₁₉-Phe₂₀ and Cys-Aβ10-25 statine Phe₂₀-Ala₂₁ and antibodies raised against Aβ₁₀₋₂₅ having a reduced amide bond between Phe₁₉ and Phe₂₀. The document provides no in vivo evidence that administration of these antibodies causes efflux of Aβ from the central nervous system, interference with plaque formation, reduction in plaque burden, formation of complexes between the antibodies and Aβ in tissue samples, or affects cognition.

U.S. Pat. Nos. 5,766,846, 5,837,672, and 5,593,846 (which are incorporated herein by reference) describe the production of murine monoclonal antibodies to the central domain of the Aβ peptide. Among antibodies known to bind between amino acids 13 and 28 of Aβ are mouse antibodies 266, 4G8, and 1 C2.

It had previously been found, as described in PCT/US/01/06191, filed Feb. 26, 2001, that administration of the mouse antibody 266 almost completely restores cognition following prolonged periods of weekly administration of the 266 antibody (object memory) in 24-month old hemizygous transgenic mice (APP^{V717F}). It was also observed that peripheral administration of antibody 266 results in rapid efflux of relatively large quantities of Aβ peptide from the CNS into the plasma. Prolonged treatment also resulted in altered clearance of soluble Aβ, prevention of plaque formation, and improvement in cognition, even without necessarily having the features the art teaches are required: for an antibody to be effective, namely, reducing Aβ amyloid plaque burden, crossing the blood brain barrier to any significant extent, decorating plaque, activating cellular mechanisms, or binding with great affinity to aggregated Aβ. DeMattos, et al. (Proc. Natl. Acad. Sci (USA) Early Edition, Jul. 3, 2001) published some of the data that are in PCT/US/01/06191. PCT/US/01/06191 also disclosed humanized 266 antibodies (“Hu266” or “h266”).

Starting at position 56 of the heavy chain V region, both Mu266 and Hu266 contain the sequence Asn-Ser-Thr. This sequence is an example of the Asn-X-Ser/Thr signal for N-linked glycosylation, wherein the Asn is the site of attachment of N-linked glycosyl chains. While most occurrences of Asn-X-Ser/Thr in secreted proteins are glycosylated (Gavel, Y. et al., Prot. Eng. (1990) 3:433-442), not all glycosylation site sequences that are present in a polypeptide are sites where sugar residues are actually attached (U.S. Pat. No. 5,714,350). Notably, the results reported in PCT/US/01/06191 were generated using a 266 antibody that was fully glycosylated at position 56 of the heavy chain.

It has been shown that glycosylation in variable region framework can have a negative effect on antibody binding affinity, likely due to steric hindrance (Co, M. S., et al., Mol. Immunol. (1993) 30:1361-1367). In contrast, glycosylation in the heavy chain CDR2 of a particular murine antibody increased its affinity for the antigen (Wallick, S.C., et al., J. Exp. Med. (1988) 168:1099-1109; Wright, A., et al., EMBO J. (1991) 10:2717-2723). In light of these teachings, the effect of glycosylation of h266 in VH CDR2 on its affinity for Aβ was unpredictable, that is, glycosylation might affect affinity for Aβ positively, negatively, or not at all. The only way to determine whether glycosylation of 266 affected affinity was to remove the glycosylation site and determine the binding affinity.

Quite unpredictably and advantageously, the affinity of Hu266 that is deglycosylated in the heavy chain CDR2 for Aβ peptide is markedly higher than that of h266.

### SUMMARY OF THE INVENTION

This invention provides humanized antibodies and fragments thereof, having the CDR of mouse anti-Aβ antibody 266, wherein the N-glycosylation site in heavy chain CDR2 is modified so that it cannot be N-glycosylated. So, in its broadest extent, the present invention is an antibody, or fragment thereof, comprising a light chain and a heavy chain, wherein the light chain comprises the three light chain complementarity determining regions (CDRs) from mouse monoclonal antibody 266 (SEQ ID NO:1-3), and wherein the heavy chain comprises heavy chain CDR1 and CDR3 from mouse monoclonal antibody 266 (SEQ ID NO:4 and 6, respectively), and a heavy chain CDR2 having the sequence given by SEQ ID NO:5:

| | |
|---|---|
| 1 5 10 15 | (SEQ ID NO:5) |
| Gln Ile Asn Ser Val Gly Xaa Xaa Xaa Tyr Tyr Pro Asp Thr Val Lys Gly | |

wherein:

Xaa at position 7 is any amino acid, provided that if Xaa at position 8 is neither Asp nor Pro and Xaa at position 9 is Ser or Thr, then Xaa at position 7 is not Asn;

Xaa at position 8 is any amino acid, provided that if Xaa at position 7 is Asn and Xaa at position 9 is Ser or Thr, then Xaa at position 8 is Asp or Pro; and

Xaa at position 9 is any amino acid, provided that if Xaa at position 7 is Asn and Xaa at position 8 is neither Asp nor Pro, then Xaa at position 9 is neither Ser nor Thr.

Also part of the invention are polynucleotide sequences that encode the humanized antibodies or fragments thereof disclosed above, vectors comprising the polynucleotide sequences encoding the humanized antibodies or fragments thereof, host cells transformed with the vectors or incorporating the polynucleotides that express the humanized antibodies or fragments thereof, pharmaceutical formulations of the humanized antibodies and fragments thereof disclosed herein, and methods of making and using the same.

Such humanized antibodies and fragments thereof having higher affinity for Aβ than mouse 266 or humanized 266 are expected to exhibit the same properties described previously for mouse 266 and humanized 266, namely, they are useful for sequestering Aβ in humans; for treating and preventing diseases and conditions characterized by Aβ plaques or Aβ toxicity in the brain, such as Alzheimer's disease, Down's syndrome, and cerebral amyloid arigiopathy in humans; for diagnosing these diseases in humans; and for determining whether a human subject will respond to treatment using humanized antibodies against Aβ.

The advantages of the present humanized, variant 266 antibodies over the previously described humanized 266 antibodies include more reliable manufacturability, less batch-to-batch variability in glycosylation, and comparable or higher affinity for the antigen than the previously described humanized 266 antibodies. This will permit lower doses to give equivalent results.

Administration of an antibody of this invention in vivo to sequester Aβ peptide circulating in biological fluids is useful for preventive and therapeutic treatment of conditions associated with the formation of Aβ-containing diffuse, neuritic, and cerebrovascular plaques in the brain. This invention provides enhanced binding affinity due to the elimination of the CDR2 N-glycosylation site.

The invention also includes methods of using the deglycosylated 266 antibodies to treat and to prevent conditions characterized by the formation of plaques containing beta-amyloid protein in humans, which method comprises administering, preferably peripherally, to a human in need of such treatment a therapeutically or prophylactically effective amount of deglycosylated 266 antibodies, or immunologically reactive fragments thereof.

In another aspect, the invention is directed to a method to inhibit the formation of amyloid plaques and to clear amyloid plaques in humans, which method comprises administering to a human subject in need of such inhibition an effective amount of the deglycosylated 266 antibodies of the present invention.

The invention also includes methods of reversing cognitive decline, improving cognitive cognition, treating cognitive decline, and preventing cognitive decline in a subject diagnosed with clinical or pre-clinical Alzheimer's disease, Down's syndrome, or clinical or pre-clinical cerebral amyloid angiopathy, comprising administering to the subject an effective amount of the deglycosylated 266 antibodies of the present invention.

The invention also includes use of a humanized antibody of the present invention for the manufacture of a medicament, including prolonged expression of recombinant sequences of the antibody or antibody fragment in human tissues, for treating, preventing, or reversing Alzheimer's disease, Down's syndrome, or cerebral amyloid angiopathy; for treating, preventing, or reversing cognitive decline in clinical or pre-clinical Alzheimer's disease, Down's syndrome, or clinical or pre-clinical cerebral amyloid angiopathy; or to inhibit the formation of amyloid plaques or the effects of toxic soluble Aβ species in humans.

### BRIEF DESCRIPTION OF THE DRAWINGS

FIG. 1. pVk-Hu266 polynucleotide sequences for expressing humanized variant 266 light chain and single amino acid codes for expressed humanized 266 light chains. The complete sequence of the light chain gene is located between the MluI and BamHI sites in pVk-Hu266. The nucleotide number indicates its position in pVk-Hu266. The Vₖ and Cₖ exons are translated in single letter code; the dot indicates the translation termination codon. The mature light chain starts at the double-underlined aspartic acid (D). The intron sequences are in italic.

FIG. 2. Complete sequence of the Hu266 N56S heavy chain gene. Complete sequence of the Hu266 N56S heavy chain gene located between the MluI and BamHI sites in pVg1-Hu266 N56S. The nucleotide number indicates its position in pVg1-Hu266 N56S. The VH and CH exons are translated in single letter code; the dot indicates the translation termination codon. The mature heavy chain starts at the bold and underlined glutamic acid (E). The adenine at nucleotide position 853 of pVg1-Hu266 has been substituted with a guanine (bold and double-underlined), resulting in an amino acid change to a serine residue (bold and double-underlined). The intron sequences are in italics. The polyA signal is underlined.

FIG. 3. Complete sequence of the Hu266 N56T heavy chain gene. Complete sequence of the Hu266 N56T heavy chain gene located between the MluI and BamHI sites in pVg1-Hu266 N56T. The nucleotide number indicates its position in pVg1-Hu266 N56T. The VH and CH exons are translated in single letter code; the dot indicates the translation termination codon. The mature heavy chain starts at the bold and underlined glutamic acid (E). The adenine at nucleotide position 853 of pVg1-Hu266 has been substituted with a cytosine (bold and double-underlined), resulting in an amino acid change to a threonine residue (bold and double-underlined). The intron sequences are in italics. The polyA signal is underlined.

FIG. 4. Nucleotide sequence and deduced amino acid sequence of the heavy chain variable region of Hu266 N56S in the mini exon. The adenine at nucleotide position 235 has been substituted with a guanine (bold and double-underlined), resulting in an amino acid change to a serine residue (bold and double-underlined). The signal peptide sequence is in italics. The CDRs based on the definition of Kabat (Johnson, J., et al., Nucleic Acids Res. (2000) 28:214-218) are underlined. The mature heavy chain begins with a glutamic acid residue (bold and underlined). The sequence shown is flanked by unique MluI (ACGCGT) and Xbal (TCTAGA) sites.

FIG. 5. Nucleotide sequence and deduced amino acid sequence of the heavy chain variable region of Hu266 N56T in the mini exon. The adenine at nucleotide position 235 has been substituted with a cytosine (bold and double-underlined), resulting in an amino acid change to a threonine residue (bold and double-underlined). The signal peptide sequence is in italics. The CDRs based on the definition of Kabat (Johnson, J., et al., Nucleic Acids Res. (2000) 28:214-218) are underlined. The mature heavy chain begins with a glutamic acid residue (bold and underlined). The sequence shown is flanked by unique MluI (ACGCGT) and XbaI (TCTAGA) sites.

FIG. 6. Hu266 N56S heavy chain cDNA and translated amino acid sequence. The amino acids are shown in single letter code; the dot indicates the translation termination codon. The first amino acid of the mature heavy chain is underlined and bold, preceded by its signal peptide sequence. The substituted amino acid, serine, is bold.

FIG. 7. Hu266 N56T heavy chain cDNA and translated amino acid sequence. The amino acids are shown in single letter code; the dot indicates the translation termination codon. The first amino acid of the mature heavy chain is underlined and bold, preceded by its signal peptide sequence. The substituted amino acid, threonine, is bold.

FIG. 8. Plasmid pVk-Hu266 FIG. 9. Plasmid construct for expression of Hu266 N56S and N56T. The Hu266 variant VH genes were constructed as mini-exons flanked by MluI and XbaI sites. The V regions were incorporated into the corresponding expression vectors to make pVg1-Hu266 N56S or N56T.

### DETAILED DESCRIPTION OF THE INVENTION

We have surprisingly found that humanized antibodies, wherein the CDRs originate from mouse monoclonal antibody 266 and the framework and other portions of the antibodies originate from a human germ line, and wherein an N-glycosylation site within the CDR2 of the heavy chain is removed, bind Aβ1-40 and Aβ1-42 with surprisingly higher affinity than glycosylated mouse or humanized 266 antibodies. Thus, we have a reasonable basis for believing that humanized antibodies of this specificity, modified to reduce their immunogenicity by converting them to a humanized form, offer the opportunity to treat, both prophylactically and therapeutically, conditions in humans that are associated with Aβ, including, pre-clinical and clinical Alzheimer's, Down's syndrome, and pre-clinical and clinical cerebral amyloid angiopathy.

As used herein, the word “treat” includes therapeutic treatment, where a condition to be treated is already known to be present and prophylaxis—i.e., prevention of, or amelioration of, the possible future onset of a condition.

By “antibody” is meant a monoclonal antibody per se, or an immunologically effective fragment thereof, such as an Fab, Fab′, or F(ab′)2 fragment thereof. In some contexts, herein, fragments will be mentioned specifically for emphasis; nevertheless, it will be understood that regardless of whether fragments are specified, the term “antibody” includes such fragments as well as single-chain forms. As long as the protein retains the ability specifically to bind its intended target, it is included within the term “antibody.” Also included within the definition “antibody” are single chain forms. Preferably, but not necessarily, the antibodies useful in the invention are produced recombinantly. Antibodies may or may not be glycosylated, though glycosylated antibodies are preferred, except at the N-glycosylation site on CDR2. Antibodies are properly cross-linked via disulfide bonds, as is well known.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. Within each isotype, there may be subtypes, such as IgG₁, IgG₄, etc. Within light and heavy chains, the variable and constant regions are joined by a “J” region of about 12 or more amino acids, with the heavy chain also including a “D” region of about 3 or more amino acids. The particular identity of constant region, the isotype, or subtype does not impact the present invention.

The variable regions of each light/heavy chain pair form the antibody binding site. Thus, an intact antibody has two binding sites. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with well known conventions [Kabat “Sequences of Proteins of Immunological Interest” National Institutes of Health, Bethesda, Md., 1987 and 1991; Chothia, et al., J. Mol. Biol. 196:901-917 (1987); Chothia, et al., Nature 342:878-883 (1989)].

By “humanized antibody” is meant an antibody that is composed partially or fully of amino acid sequences derived from a human antibody germline by altering the sequence of an antibody having non-human complementarity determining regions (CDR). A humanized immunoglobulin does not encompass a chimeric antibody, having a mouse variable region and a human constant region. However, the variable region of the antibody and even the CDR are humanized by techniques that are by now well known in the art. The framework regions of the variable regions are substituted by the corresponding human framework regions leaving the non-human CDR substantially intact. As mentioned above, it is sufficient for use in the methods of the invention, to employ an immunologically specific fragment of the antibody, including fragments representing single chain forms.

Humanized antibodies have at least three potential advantages over non-human and chimeric antibodies for use in human therapy:

1) because the effector portion is human, it may interact better with the other parts of the human immune system (e.g., destroy the target cells more efficiently by complement-dependent cytotoxicity (CDC) or antibody-dependent cellular cytotoxicity (ADCC)).

2) The human immune system should not recognize the framework or C region of the humanized antibody as foreign, and therefore the antibody response against such an injected antibody should be less than against a totally foreign non-human antibody or a partially foreign chimeric antibody.

3) Injected non-human antibodies have been reported to have a half-life in the human circulation much shorter than the half-life of human antibodies. Injected humanized antibodies will have a half-life essentially identical to naturally occurring human antibodies, allowing smaller and less frequent doses to be given.

The design of humanized immunoglobulins may be carried out as follows. As to the human framework region, a framework or variable region amino acid sequence of a CDR-providing non-human immunoglobulin is compared with corresponding sequences in a human immunoglobulin variable region sequence collection, and a sequence having a high percentage of identical amino acids is selected. When an amino acid falls under the following category, the framework amino acid of a human immunoglobulin to be used (acceptor immunoglobulin) is replaced by a framework amino acid from a CDR-providing non-human immunoglobulin (donor immunoglobulin):

(a) the amino acid in the human framework region of the acceptor immunoglobulin is unusual for human immunoglobulin at that position, whereas the corresponding amino acid in the donor immunoglobulin is typical for human immunoglobulin at that position;

(b) the position of the amino acid is immediately adjacent to one of the CDRs; or

(c) any side chain atom of a framework amino acid is within about 5-6 angstroms (center-to-center) of any atom of a CDR amino acid in a three dimensional immunoglobulin model [Queen, et al., Proc. Natl Acad. Sci. USA 86:10029-10033 (1989), and Co, et al., Proc. Natl. Acad. Sci. USA 88, 2869 (1991)]. When each of the amino acid in the human framework region of the acceptor immunoglobulin and a corresponding amino acid in the donor immunoglobulin is unusual for human immunoglobulin at that position, such an amino acid is replaced by an amino acid typical for human immunoglobulin at that position.

The CDRs of deglycosylated humanized 266 have the following amino acid sequences:

| | |
|---|---|
| light chain CDR1: | |
| 1 5 10 15 | (SEQ ID NO:1) |
| Arg Ser Ser Gln Ser Leu Ile Tyr Ser Asp Gly Asn Ala Tyr Leu His | |
| light chain CDR2: | |
| 1 5 | (SEQ ID NO:2) |
| Lys Val Ser Asn Arg Phe Ser | |
| light chain CDR3: | |
| 1 5 | (SEQ ID NO:3) |
| Ser Gln Ser Thr His Val Pro Trp Thr | |
| heavy chain CDR1: | |
| 1 5 | (SEQ ID NO:4) |
| Arg Tyr Ser Met Ser | |
| heavy chain CDR2: | |
| 1 5 10 15 | (SEQ ID NO:5) |
| Gln Ile Asn Ser Val Gly Xaa Xaa Xaa Tyr Tyr Pro Asp Thr Val Lys Gly | |

wherein:

Xaa at position 7 is any amino acid, provided that if Xaa at position 8 is neither Asp nor Pro and Xaa at position 9 is Ser or Thr, then Xaa at position 7 is not Asn;

Xaa at position 8 is any amino acid, provided that if Xaa at position 7 is Asn and Xaa at position 9 is Ser or Thr, then Xaa at position 8 is Asp or Pro; and

Xaa at position 9 is any amino acid, provided that if Xaa at position 7 is Asn and Xaa at position 8 is neither Asp nor Pro, then Xaa at position 9 is neither Ser nor Thr; and, heavy chain CDR3:

| | |
|---|---|
| 1 | (SEQ ID NO:6) |
| Gly Asp Tyr. | |

By “any amino acid” is meant any naturally-occurring amino acid. Preferred naturally-occurring amino acids are Ala, Cys, Asp, Glu, Phe, Gly, His, Ile, Lys, Leu, Met, Asn, Pro, Gln, Arg, Ser, Thr, Val, Trp, and Tyr.

A preferred group of antibodies are those having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is SEQ ID NO:5, wherein:

Xaa at position 7 of SEQ ID NO:5 is selected from the group consisting of Ala, Cys, Asp, Glu, Phe, Gly, His, Ile, Lys, Leu, Met, Asn, Pro, Gln, Arg, Ser, Thr, Val, Trp, and Tyr, provided that if Xaa at position 8 is neither Asp nor Pro and Xaa at position 9 is Ser or Thr, then Xaa at position 7 is not Asn;

Xaa at position 8 of SEQ ID NO:5 is selected from the group consisting of Ala, Cys, Asp, Glu, Phe, Gly, His, Ile, Lys, Leu, Met, Asn, Pro, Gin, Arg, Ser, Thr, Val, Trp, and Tyr, provided that if Xaa at position 7 is Asn and Xaa at position 9 is Ser or Thr, then Xaa at position 8 is Asp or Pro; and

Xaa at position 9 of SEQ ID NO:5 is selected from the group consisting of Ala, Cys, Asp, Glu, Phe, Gly, His, Ile, Lys, Leu, Met, Asn, Pro, Gin, Arg, Ser, Thr, Val, Trp, and Tyr, provided that if Xaa at position 7 is Asn and Xaa at position 8 is neither Asp nor Pro, then Xaa at position 9 is neither Ser nor Thr.

Another description of the preferred group is: antibodies or fragments thereof having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is selected from the group consisting of:

| | |
|---|---|
| 1) SEQ ID NO:13 | |
| 1 5 10 15 | (SEQ ID NO:13) |
| Gln Ile Asn Ser Val Gly Xaa Xaa Xaa Tyr Tyr Pro Asp Thr Val Lys Gly | |

wherein:

Xaa at position 7 of SEQ ID NO:13 is selected from the group consisting of Ala, Cys, Asp, Glu, Phe, Gly, His, Ile, Lys, Leu, Met, Pro, Gin, Arg, Ser, Thr, Val, Trp, and Tyr;

Xaa at position 8 of SEQ ID NO:13 is selected from the group consisting of Ala, Cys, Asp, Glu, Phe, Gly, His, Ile, Lys, Leu, Met, Asn, Pro, Gin, Arg, Ser, Thr, Val, Trp, and Tyr; and

Xaa at position 9 of SEQ ID NO:13 is selected from the group consisting of Ala, Cys, Asp, Glu, Phe, Gly, His, Ile, Lys, Leu, Met, Asn, Pro, Gin, Arg, Ser, Thr, Val, Trp, and Tyr;

| | |
|---|---|
| 2) SEQ ID NO:14 | |
| 1 5 10 15 | (SEQ ID NO:14) |
| Gln Ile Asn Ser Val Gly Xaa Xaa Xaa Tyr Tyr Pro Asp Thr Val Lys Gly | |

wherein:

Xaa at position 7 of SEQ ID NO:14 is Asn;

Xaa at position 8 of SEQ ID NO:14 is selected from the group consisting of Ala, Cys, Asp, Glu, Phe, Gly, His, Ile, Lys, Leu, Met, Asn, Pro, Gln, Arg, Ser, Thr, Val, Trp, and Tyr; and

Xaa at position 9 of SEQ ID NO:14 is selected from the group consisting of Ala, Cys, Asp, Glu, Phe, Gly, His, Ile, Lys, Leu, Met, Asn, Pro, Gln, Arg, Val, Trp, and Tyr;

and

| | |
|---|---|
| 3) SEQ ID NO:15 | |
| 1 5 10 15 | (SEQ ID NO:15) |
| Gln Ile Asn Ser Val Gly Xaa Xaa Xaa Tyr Tyr Pro Asp Thr Val Lys Gly | |

wherein:

Xaa at position 7 of SEQ ID NO:15 is Asn;

Xaa at position 8 of SEQ ID NO:15 is selected from the group consisting of Asp and Pro; and

Xaa at position 9 of SEQ ID NO:15 is selected from the group consisting of Ser and Thr.

Preferred sequences for CDR2 of the heavy chain include those in which only a single amino acid is changed, those in which only two amino acids are changed, or all three are changed. It is preferred to replace Asn at position 7, or to replace Thr at position 9, or to replace both. Conservative substitutions at one, two, or all three positions are preferred. The most preferred species are those in which Asn at position 7 is replaced with Ser or Thr. It is preferred to not replace Ser at position 8, and if Ser at position 8 is replaced, then to replace it conservatively, for example, with Ala or Thr.Preferred deglycosylated 266 antibodies of the present invention are those in which in CDR2 of the heavy chain (i.e., within SEQ ID NO:5, as described above):

Xaa at position 7 is selected from the group consisting of Ala, Gly, His, Asn, Gln, Ser, and Thr, provided that if Xaa at position 9 is Ser or Thr, then Xaa at position 7 is not Asn;

Xaa at position 8 is selected from the group consisting of Ala, Gly, His, Asn, Gln, Ser, and Thr; and

Xaa at position 9 is selected from the group consisting of Ala, Gly, His, Asn, Gln, Ser, and Thr, provided that if Xaa at position 7 is Asn, then Xaa at position 9 is neither Ser nor Thr.

An alternate description of preferred deglycogsylated 266 antibodies is: antibodies or fragments thereof having as light chain CDR1-CDR3 the sequences SEQ ID NO:1-3, respectively, as heavy chain CDR1 and CDR3 the sequences SEQ ID NO:4 and 6, respectively, and wherein the sequence of heavy chain CDR2 is selected from the group consisting of:

| | |
|---|---|
| 1) SEQ ID NO:16 | |
| 1 5 10 15 | (SEQ ID NO:16) |
| Gln Ile Asn Ser Val Gly Xaa Xaa Xaa Tyr Tyr Pro Asp Thr Val Lys Gly | |

wherein:

Xaa at position 7 of SEQ ID NO:16 is selected from the group consisting of Ala, Gly, His, Gln, Ser, and Thr;

Xaa at position 8 of SEQ ID NO:16 is selected from the group consisting of Ala, Gly, His, Asn, Gln, Ser, and Thr; and

Xaa at position 9 of SEQ ID NO:16 is selected from the group consisting of Ala, Gly, His, Asn, Gln, Ser, and Thr; and

| | |
|---|---|
| 2) SEQ ID NO:17 | |
| 1 5 10 15 | (SEQ ID NO:17) |
| Gln Ile Asn Ser Val Gly Xaa Xaa Xaa Tyr Tyr Pro Asp Thr Val Lys Gly | |

wherein:

Xaa at position 7 of SEQ ID NO:17 is Asn;

Xaa at position 8 of SEQ ID NO:17 is selected from the group consisting of Ala, Gly, His, Asn, Gln, Ser, and Thr; and

Xaa at position 9 of SEQ ID NO:17 is selected from the group consisting of Ala, Gly, His, Asn, and Gln.

Another group of preferred deglycosylated 266 antibodies are those in which in CDR2 of the heavy chain (i.e., within SEQ ID NO:5, as described above):

Xaa at position 7 is selected from the group consisting of Ala, Gly, Leu, Met, Gln, Ser, Thr, and Val;

Xaa at position 8 is Ser; and

Xaa at position 9 is Thr.

Another group of preferred deglycosylated 266 antibodies are those in which in CDR2 of the heavy chain (i.e., within SEQ ID NO:5, as described above):

Xaa at position 7 is Asn;

Xaa at position 8 is Ser; and

Xaa at position 9 is selected from the group consisting of Ala, Gly, Asn, Gln, and Val.

Another group of preferred deglycosylated 266 antibodies are those in which in CDR2 of the heavy chain (i.e., within SEQ ID NO:5, as described above):

Xaa at position 7 is selected from the group consisting of Ala, Gly, Leu, Met, Gln, Ser, Thr, and Val;

Xaa at position 8 is Ser; and

Xaa at position 9 is selected from the group consisting of Ala, Gly, Asn, Gln, and Val.

Another group of preferred deglycosylated 266 antibodies are those in which in CDR2 of the heavy chain (i.e., within SEQ ID NO:5, as described above):

Xaa at position 7 is selected from the group consisting of Ser and Thr;

Xaa at position 8 is selected from the group consisting of Ser, Ala, and Thr; and

Xaa at position 9 is selected from the group consisting of Ala, Gly, Asn, Gln, Thr, and Val.

Another group of preferred deglycosylated 266 antibodies are those in which in CDR2 of the heavy chain (i.e., within SEQ ID NO:5, as described above):

Xaa at position 7 is selected from the group consisting of Ser and Thr;

Xaa at position 8 is selected from the group consisting of Ser, Ala, and Thr; and

Xaa at position 9 is Thr.

A preferred light chain variable region of a humanized antibody of the present invention has the following amino acid sequence, in which the framework originated from human germline Vk segment DPK18 and J segment Jk1, with several amino acid substitutions to the consensus amino acids in the same human V subgroup to reduce potential immunogenicity:

| | |
|---|---|
| 1 5 10 15 | (SEQ ID NO:7) |
| Asp Xaa Val Met Thr Gln Xaa Pro Leu Ser Leu Pro Val Xaa Xaa | |
| 20 25 30 | |
| Gly Gln Pro Ala Ser Ile Ser Cys Arg Ser Ser Gln Ser Leu Xaa | |
| 35 40 45 | |
| Tyr Ser Asp Gly Asn Ala Tyr Leu His Trp Phe Leu Gln Lys Pro | |
| 50 55 60 | |
| Gly Gln Ser Pro Xaa Leu Leu Ile Tyr Lys Val Ser Asn Arg Phe | |
| 65 70 75 | |
| Ser Gly Val Pro Asp Arg Phe Ser Gly Ser Gly Ser Gly Thr Asp | |
| 80 85 90 | |
| Phe Thr Leu Lys Ile Ser Arg Val Glu Ala Glu Asp Xaa Gly Val | |
| 95 100 105 | |
| Tyr Tyr Cys Ser Gln Ser Thr His Val Pro Trp Thr Phe Gly Xaa | |
| 110 | |
| Gly Thr Xaa Xaa Glu Ile Lys Arg | |

wherein:

Xaa at position 2 is Val or Ile;

Xaa at position 7 is Ser or Thr;

Xaa at position 14 is Thr or Ser;

Xaa at position 15 is Leu or Pro;

Xaa at position 30 is Ile or Val;

Xaa at position 50 is Arg, Gln, or Lys;

Xaa at position 88 is Val or Leu;

Xaa at position 105 is Gln or Gly;

Xaa at position 108 is Lys or Arg; and

Xaa at position 109 is Val or Leu.

A preferred heavy chain variable region of a humanized antibody of the present invention has the following amino acid sequence, in which the framework originated from human germline VH segment DP53 and J segment JH4, with several amino acid substitutions to the consensus amino acids in the same human subgroup to reduce potential immunogenicity:

| | |
|---|---|
| 1 5 10 15 | (SEQ ID NO:8) |
| Xaa Val Gln Leu Val Glu Xaa Gly Gly Gly Leu Val Gln Pro Gly | |
| 20 25 30 | |
| Gly Ser Leu Arg Leu Ser Cys Ala Ala Ser Gly Phe Thr Phe Ser | |
| 35 40 45 | |
| Arg Tyr Ser Met Ser Trp Val Arg Gln Ala Pro Gly Lys Gly Leu | |
| 50 55 60 | |
| Xaa Leu Val Ala Gln Ile Asn Ser Val Gly Xaa Xaa Xaa Tyr Tyr | |
| 65 70 75 | |
| Pro Asp Xaa Val Lys Gly Arg Phe Thr Ile Ser Arg Asp Asn Xaa | |
| 80 85 90 | |
| Xaa Asn Thr Leu Tyr Leu Gln Met Asn Ser Leu Arg Ala Xaa Asp | |
| 95 100 105 | |
| Thr Ala Val Tyr Tyr Cys Ala Ser Gly Asp Tyr Trp Gly Gln Gly | |
| 110 | |
| Thr Xaa Val Thr Val Ser Ser | |

wherein:

Xaa at position 1 is Glu or Gln;

Xaa at position 7 is Ser or Leu;

Xaa at position 46 is Glu, Val, Asp, or Ser;

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr

Xaa at position 63 is Thr or Ser;

Xaa at position 75 is Ala, Ser, Val, or Thr;

Xaa at position 76 is Lys or Arg;

Xaa at position 89 is Glu or Asp; and

Xaa at position 107 is Leu or Thr.

A particularly preferred light chain variable region of a humanized antibody of the present invention has the following amino acid sequence, in which the framework originated from human germline Vk segment DPK18 and J segment Jk1, with several amino acid substitutions to the consensus amino acids in the same human V subgroup to reduce potential immunogenicity:

| | |
|---|---|
| 1 5 10 15 | (SEQ ID NO:9) |
| Asp Val Val Met Thr Gln Ser Pro Leu Ser Leu Pro Val Thr Leu | |
| 20 25 30 | |
| Gly Gln Pro Ala Ser Ile Ser Cys Arg Ser Ser Gln Ser Leu Ile | |
| 35 40 45 | |
| Tyr Ser Asp Gly Asn Ala Tyr Leu His Trp Phe Leu Gln Lys Pro | |
| 50 55 60 | |
| Gly Gln Ser Pro Arg Leu Leu Ile Tyr Lys Val Ser Asn Arg Phe | |
| 65 70 75 | |
| Ser Gly Val Pro Asp Arg Phe Ser Gly Ser Gly Ser Gly Thr Asp | |
| 80 85 90 | |
| Phe Thr Leu Lys Ile Ser Arg Val Glu Ala Glu Asp Val Gly Val | |
| 95 100 105 | |
| Tyr Tyr Cys Ser Gln Ser Thr His Val Pro Trp Thr Phe Gly Gln | |
| 110 | |
| Gly Thr Lys Val Glu Ile Lys Arg. | |

A particularly preferred heavy chain variable region of a humanized antibody of the present invention has the following amino acid sequence, in which the framework originated from human germline VH segment DP53 and J segment JH4:

| | |
|---|---|
| 1 5 10 15 | (SEQ ID NO:10) |
| Glu Val Gln Leu Val Glu Ser Gly Gly Gly Leu Val Gln Pro Gly | |
| 20 25 30 | |
| Gly Ser Leu Arg Leu Ser Cys Ala Ala Ser Gly Phe Thr Phe Ser | |
| 35 40 45 | |
| Arg Tyr Ser Met Ser Trp Val Arg Gln Ala Pro Gly Lys Gly Leu | |
| 50 55 60 | |
| Glu Leu Val Ala Gln Ile Asn Ser Val Gly Xaa Xaa Xaa Tyr Tyr | |
| 65 70 75 | |
| Pro Asp Thr Val Lys Gly Arg Phe Thr Ile Ser Arg Asp Asn Ala | |
| 80 85 90 | |
| Lys Asn Thr Leu Tyr Leu Gln Met Asn Ser Leu Arg Ala Glu Asp | |
| 95 100 105 | |
| Thr Ala Val Tyr Tyr Cys Ala Ser Gly Asp Tyr Trp Gly Gln Gly | |
| 110 | |
| Thr Leu Val Thr Val Ser Ser | |

wherein:

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr.

A preferred light chain for a humanized antibody of the present invention has the amino acid sequence:

| | |
|---|---|
| 1 5 10 15 | (SEQ ID NO:11} |
| Asp Val Val Met Thr Gln Ser Pro Leu Ser Leu Pro Val Thr Leu | |
| 20 25 30 | |
| Gly Gln Pro Ala Ser Ile Ser Cys Arg Ser Ser Gln Ser Leu Ile | |
| 35 40 45 | |
| Tyr Ser Asp Gly Asn Ala Tyr Leu His Trp Phe Leu Gln Lys Pro | |
| 50 55 60 | |
| Gly Gln Ser Pro Arg Leu Leu Ile Tyr Lys Val Ser Asn Arg Phe | |
| 65 70 75 | |
| Ser Gly Val Pro Asp Arg Phe Ser Gly Ser Gly Ser Gly Thr Asp | |
| 80 85 90 | |
| Phe Thr Leu Lys Ile Ser Arg Val Glu Ala Glu Asp Val Gly Val | |
| 95 100 105 | |
| Tyr Tyr Cys Ser Gln Ser Thr His Val Pro Trp Thr Phe Gly Gln | |
| 110 115 120 | |
| Gly Thr Lys Val Glu Ile Lys Arg Thr Val Ala Ala Pro Ser Val | |
| 125 130 135 | |
| Phe Ile Phe Pro Pro Ser Asp Glu Gln Leu Lys Ser Gly Thr Ala | |
| 140 145 150 | |
| Ser Val Val Cys Leu Leu Asn Asn Phe Tyr Pro Arg Glu Ala Lys | |
| 155 160 165 | |
| Val Gln Trp Lys Val Asp Asn Ala Leu Gln Ser Gly Asn Ser Gln | |
| 170 175 180 | |
| Glu Ser Val Thr Glu Gln Asp Ser Lys Asp Ser Thr Tyr Ser Leu | |
| 185 190 195 | |
| Ser Ser Thr Leu Thr Leu Ser Lys Ala Asp Tyr Glu Lys His Lys | |
| 200 205 210 | |
| Val Tyr Ala Cys Glu Val Thr His Gln Gly Leu Ser Ser Pro Val | |
| 215 | |
| Thr Lys Ser Phe Asn Arg Gly Glu Cys. | |

A preferred heavy chain for a humanized antibody of the present invention has the amino acid sequence:

| | |
|---|---|
| 1 5 10 15 | (SEQ ID NO:12) |
| Glu Val Gln Leu Val Glu Ser Gly Gly Gly Leu Val Gln Pro Gly | |
| 20 25 30 | |
| Gly Ser Leu Arg Leu Ser Cys Ala Ala Ser Gly Phe Thr Phe Ser | |
| 35 40 45 | |
| Arg Tyr Ser Met Ser Trp Val Arg Gln Ala Pro Gly Lys Gly Leu | |
| 50 55 60 | |
| Glu Leu Val Ala Gln Ile Asn Ser Val Gly Xaa Xaa Xaa Tyr Tyr | |
| 65 70 75 | |
| Pro Asp Thr Val Lys Gly Arg Phe Thr Ile Ser Arg Asp Asn Ala | |
| 80 85 90 | |
| Lys Asn Thr Leu Tyr Leu Gln Met Asn Ser Leu Arg Ala Glu Asp | |
| 95 100 105 | |
| Thr Ala Val Tyr Tyr Cys Ala Ser Gly Asp Tyr Trp Gly Gln Gly | |
| 110 115 120 | |
| Thr Leu Val Thr Val Ser Ser Ala Ser Thr Lys Gly Pro Ser Val | |
| 125 130 135 | |
| Phe Pro Leu Ala Pro Ser Ser Lys Ser Thr Ser Gly Gly Thr Ala | |
| 140 145 150 | |
| Ala Leu Gly Cys Leu Val Lys Asp Tyr Phe Pro Glu Pro Val Thr | |
| 155 160 165 | |
| Val Ser Trp Asn Ser Gly Ala Leu Thr Ser Gly Val His Thr Phe | |
| 170 175 180 | |
| Pro Ala Val Leu Gln Ser Ser Gly Leu Tyr Ser Leu Ser Ser Val | |
| 185 190 195 | |
| Val Thr Val Pro Ser Ser Ser Leu Gly Thr Gln Thr Tyr Ile Cys | |
| 200 205 210 | |
| Asn Val Asn His Lys Pro Ser Asn Thr Lys Val Asp Lys Lys Val | |
| 215 220 225 | |
| Glu Pro Lys Ser Cys Asp Lys Thr His Thr Cys Pro Pro Cys Pro | |
| 230 235 240 | |
| Ala Pro Glu Leu Leu Gly Gly Pro Ser Val Phe Leu Phe Pro Pro | |
| 245 250 255 | |
| Lys Pro Lys Asp Thr Leu Met Ile Ser Arg Thr Pro Glu Val Thr | |
| 260 265 270 | |
| Cys Val Val Val Asp Val Ser His Glu Asp Pro Glu Val Lys Phe | |
| 275 280 285 | |
| Asn Trp Tyr Val Asp Gly Val Glu Val His Asn Ala Lys Thr Lys | |
| 290 295 300 | |
| Pro Arg Glu Glu Gln Tyr Asn Ser Thr Tyr Arg Val Val Ser Val | |
| 305 310 315 | |
| Leu Thr Val Leu His Gln Asp Trp Leu Asn Gly Lys Glu Tyr Lys | |
| 320 325 330 | |
| Cys Lys Val Ser Asn Lys Ala Leu Pro Ala Pro Ile Glu Lys Thr | |
| 335 340 345 | |
| Ile Ser Lys Ala Lys Gly Gln Pro Arg Glu Pro Gln Val Tyr Thr | |
| 350 355 360 | |
| Leu Pro Pro Ser Arg Asp Glu Leu Thr Lys Asn Gln Val Ser Leu | |
| 365 370 375 | |
| Thr Cys Leu Val Lys Gly Phe Tyr Pro Ser Asp Ile Ala Val Glu | |
| 380 385 390 | |
| Trp Glu Ser Asn Gly Gln Pro Glu Asn Asn Tyr Lys Thr Thr Pro | |
| 395 400 405 | |
| Pro Val Leu Asp Ser Asp Gly Ser Phe Phe Leu Tyr Ser Lys Leu | |
| 410 415 420 | |
| Thr Val Asp Lys Ser Arg Trp Gln Gln Gly Asn Val Phe Ser Cys | |
| 425 430 435 | |
| Ser Val Met His Glu Ala Leu His Asn His Tyr Thr Gln Lys Ser | |
| 440 | |
| Leu Ser Leu Ser Pro Gly Lys | |

wherein:

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr.

Preferred deglycosylated 266 antibodies having the heavy variable region according to SEQ ID NO:8, SEQ ID NO:10, and SEQ ID NO:12 are those wherein:

Xaa at position 56 is selected from the group consisting of Ala, Gly, His, Asn, Gin, Ser, and Thr, provided that if Xaa at position 58 is Ser or Thr, then Xaa at position 56 is not Asn;

Xaa at position 57 is selected from the group consisting of Ala, Gly, His, Asn, Gln, Ser, and Thr; and

Xaa at position 58 is selected from the group consisting of Ala, Gly, His, Asn, Gin, Ser, and Thr, provided that if Xaa at position 56 is Asn, then Xaa at position 58 is neither Ser nor Thr.

Preferred sequences for CDR2 (positions 56, 57, and 58) of the heavy chain SEQ ID NO:8, SEQ ID NO:10, and SEQ ID NO:12 include those in which only a single amino acid is changed, those in which only two amino acids are changed, or all three are changed. It is preferred to replace Asn at position 56. It is preferred to replace Thr at position 58 with an amino acid other than Ser. It is preferred to destroy the N-glycosylation site in the CDR2 of the 266 heavy chain by means other than replacing Ser at position 57 with Pro or Asp. Conservative substitutions at one, two, or all three positions are preferred. The most preferred species are those in which Asn at position 56 is replaced with Ser or Thr. Particularly preferred antibodies are those in which Ser or Thr is at position 56, Ser is at position 57, and Thr is at position 58 of SEQ ID NO:8, SEQ ID NO:10, or SEQ ID NO:12.

The most preferred species are antibodies comprising a light chain of SEQ ID NO:11 and a heavy chain of SEQ ID NO:12, wherein in SEQ ID NO:12, Xaa at position 56 is Ser, Xaa at position 57 is Ser, and Xaa at position 58 is Thr (“N56S”), or wherein in SEQ ID NO:12, Xaa at position 56 is Thr, Xaa at position 57 is Ser, and Xaa at position 58 is Thr (“N56T”).

Other sequences are possible for the light and heavy chains for the humanized antibodies of the present invention and for humanized 266. The immunoglobulins can have two pairs of light chain/heavy chain complexes, at least one chain comprising one or more mouse complementarity determining regions functionally joined to human framework region segments.

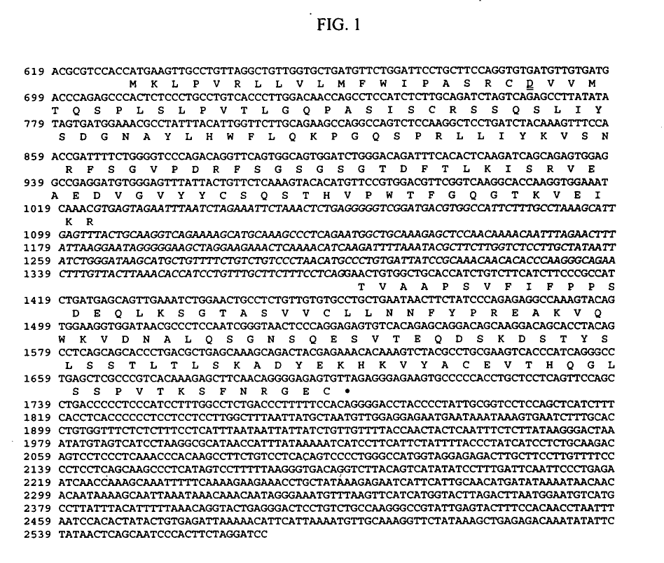

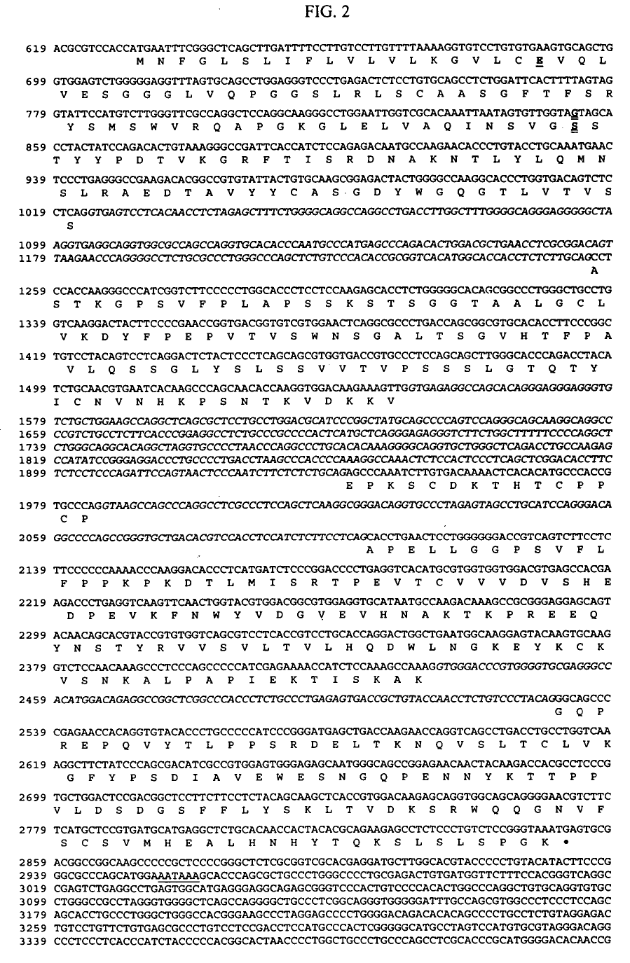

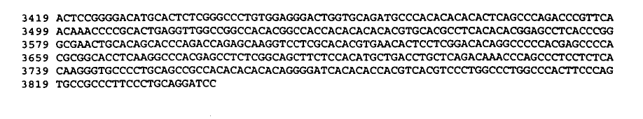

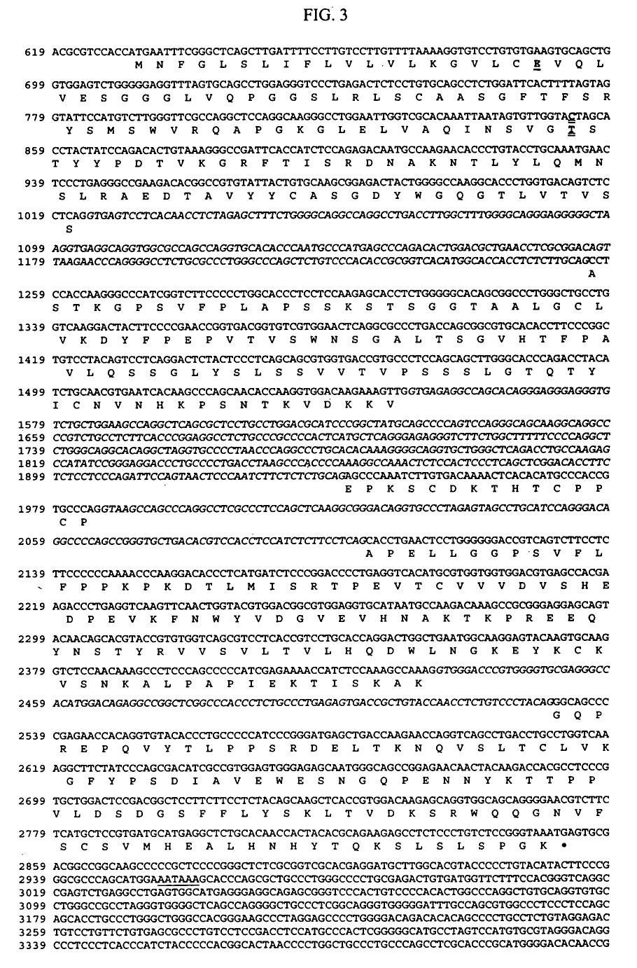

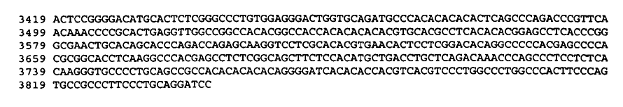

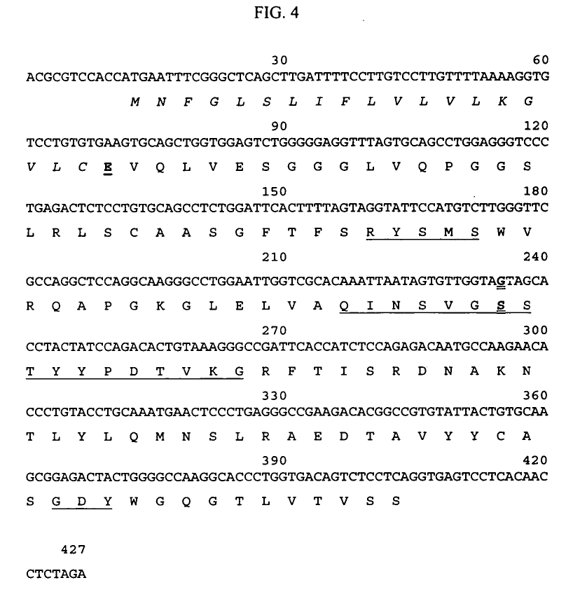

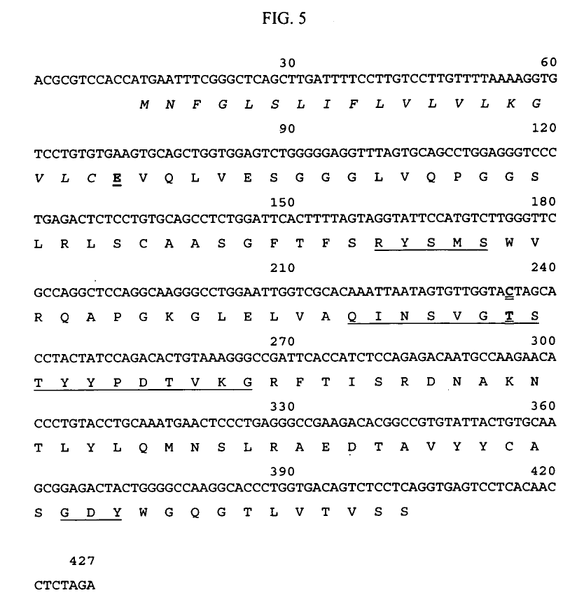

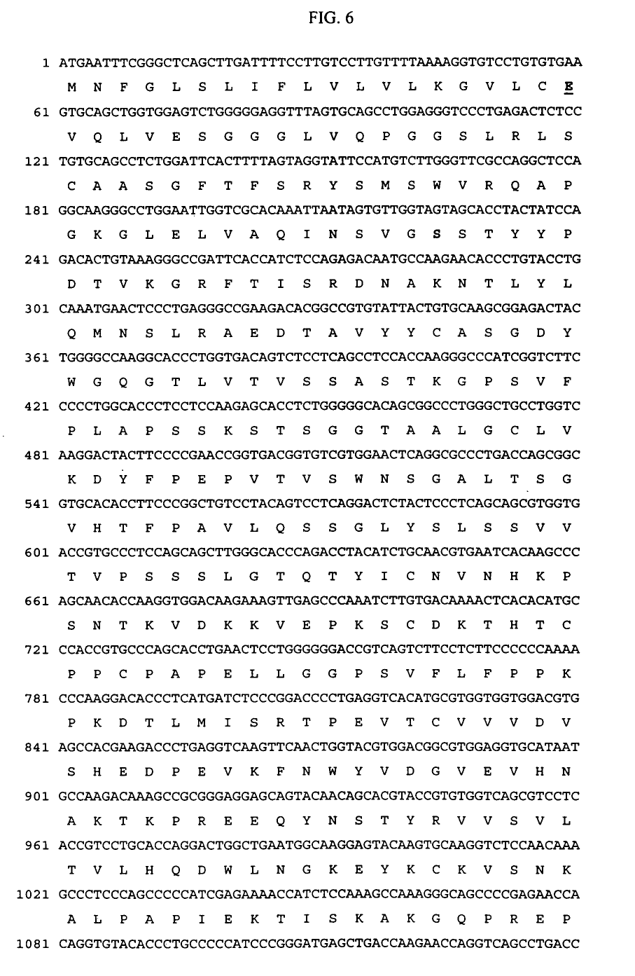

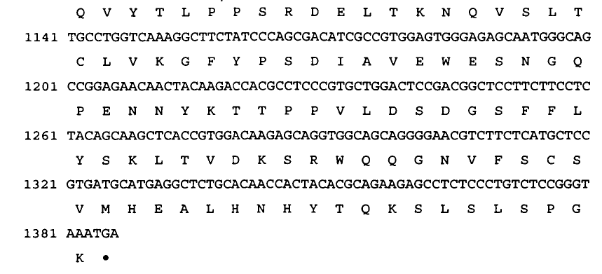

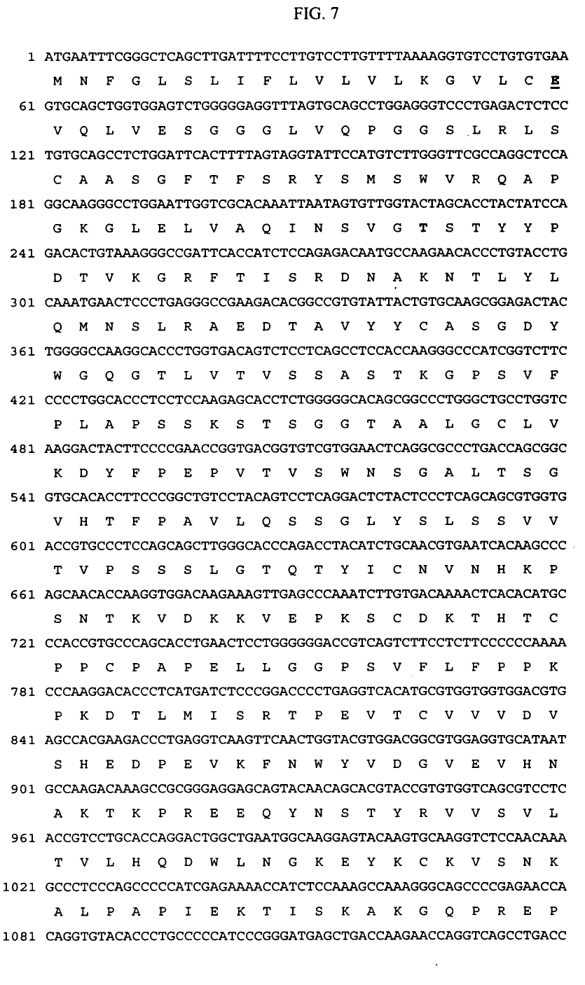

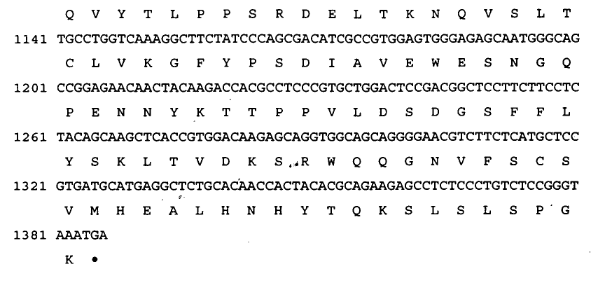

In another aspect, the present invention is directed to recombinant polynucleotides encoding antibodies which, when expressed, comprise the heavy and light chain CDRs from an antibody of the present invention. Exemplary polynucleotides, which on expression code for the polypeptide chains comprising the heavy and light chain CDRs of the present invention are given in FIGS. 1-7. Reversal of the noted heavy chain changes (FIGS. 2-6) that produce humanized antibody 266 variants N56S and N56T provides humanized antibody 266 with the CDR2 N-glycosylation site intact. Due to codon degeneracy, other polynucleotide sequences can be readily substituted for those sequences. Particularly preferred polynucleotides of the present invention encode antibodies, which when expressed, comprise the CDRs of SEQ ID NO:1-4 and 6, and SEQ ID NO:5, 13, 14, 15, 16 or 17, or any of the variable regions of SEQ ID NO:7-SEQ ID NO:10, or the light and heavy chains of SEQ ID NO:11 and SEQ ID NO:12.

The polynucleotides will typically further include an expression control polynucleotide sequence operably linked to the humanized immunoglobulin coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, the collection and purification of the light chains, heavy chains, light/heavy chain dimers or intact antibodies, binding fragments or other immunoglobulin forms may follow.

The nucleic acid sequences of the present invention capable of ultimately expressing the desired humanized antibodies can be formed from a variety of different polynucleotides (genomic or cDNA, RNA, synthetic oligonucleotides, etc.) and components (e.g., V, J, D, and C regions), using any of a variety of well known techniques. Joining appropriate genomic and synthetic sequences is a common method of production, but cDNA sequences may also be utilized.

Human constant region DNA sequences can be isolated in accordance with well known procedures from a variety of human cells, but preferably from immortalized B-cells. Suitable source cells for the polynucleotide sequences and host cells for immunoglobulin expression and secretion can be obtained from a number of sources well-known in the art.

In addition to the humanized immunoglobulins specifically described herein, other “substantially homologous” modified immunoglobulins can be readily designed and manufactured utilizing various recombinant DNA techniques well known to those skilled in the art. For example, the framework regions can vary from the native sequences at the primary structure level by several amino acid substitutions, terminal and intermediate additions and deletions, and the like. Moreover, a variety of different human framework regions may be used singly or in combination as a basis for the humanized immunoglobulins of the present invention. In general, modifications of the genes may be readily accomplished by a variety of well-known techniques, such as site-directed mutagenesis.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors using site-directed mutagenesis, such as after CH1 to produce Fab fragments or after the hinge region to produce F(ab′)2 fragments. Single chain antibodies may be produced by joining VL and VH with a DNA linker.

As stated previously, the polynucleotides will be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, cytomegalovirus and the like.

The vectors containing the polynucleotide sequences of interest (e.g., the heavy and light chain encoding sequences and expression control sequences) can be transferred into the host cell by well-known methods, which vary depending on the type of cellular host. A variety of hosts may be employed to express the antibodies of the present invention using techniques well known in the art. Mammalian tissue cell culture is preferred, especially using, for example, CHO, COS, Syrian Hamster Ovary, HeLa, myeloma, transformed B-cells, human embryonic kidney, or hybridoma cell lines.

Once expressed, the antibodies can be purified according to standard procedures. Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically or prophylactically, as directed herein.

The antibodies (including immunologically reactive fragments) are administered to a subject at risk for or exhibiting AP-related symptoms or pathology such as clinical or pre-clinical Alzheimer's disease, Down's syndrome, or clinical or pre-clinical amyloid angiopathy, using standard administration techniques, preferably peripherally (i.e. not by administration into the central nervous system) by intravenous, intraperitoneal, subcutaneous, pulmonary, transdermal, intramuscular, intranasal, buccal, sublingual, or suppository administration. Although the antibodies may be administered directly into the ventricular system, spinal fluid, or brain parenchyma, and techniques for addressing these locations are well known in the art, it is not necessary to utilize these more difficult procedures. The antibodies of the invention are effective when administered by the more simple techniques that rely on the peripheral circulation system. The advantages of the present invention include the ability of the antibody to exert its beneficial effects even though not provided directly to the central nervous system itself. In addition, humanized antibodies used in the invention, when administered peripherally, do not need to elicit a cellular immune response in brain when bound to Aβ peptide or when freely circulating to have their beneficial effects. Further, when administered peripherally they do not need to appreciably bind aggregated Aβ peptide in the brain to have their beneficial effects. Indeed, it has been demonstrated that the amount of antibody that crosses the blood-brain barrier is <0.1% of plasma levels.

The pharmaceutical compositions for administration are designed to be appropriate for the selected mode of administration, and pharmaceutically acceptable excipients such as, buffers, surfactants, preservatives, solubilizing agents, isotonicity agents, stabilizing agents and the like are used as appropriate. Remington's Pharmaceutical Sciences, Mack Publishing Co., Easton Pa., latest edition, incorporated herein by reference, provides a compendium of formulation techniques as are generally known to practitioners.

The concentration of the humanized antibody in formulations from as low as about 0.1% to as much as 15 or 20% by weight and will be selected primarily based on fluid volumes, viscosities, and so forth, in accordance with the particular mode of administration selected. Thus, a pharmaceutical composition for injection could be made up to contain in 1 mL of phosphate buffered saline from 1 to 100 mg of the humanized antibody of the present invention. The formulation could be sterile filtered after making the formulation, or otherwise made microbiologically acceptable. A typical composition for intravenous infusion could have a volume as much as 250 mL of fluid, such as sterile Ringer's solution, and 1-100 mg per mL, or more in antibody concentration. Therapeutic agents of the invention can be frozen or lyophilized for storage and reconstituted in a suitable sterile carrier prior to use. Lyophilization and reconstitution can lead to varying degrees of antibody activity loss (e.g. with conventional immune globulins, IgM antibodies tend to have greater activity loss than IgG antibodies). Dosages may have to be adjusted to compensate. The pH of the formulation will be selected to balance antibody stability (chemical and physical) and comfort to the patient when administered. Generally, pH between 4 and 8 is tolerated.

Although the foregoing methods appear the most convenient and most appropriate for administration of proteins such as humanized antibodies, by suitable adaptation, other techniques for administration, such as transdermal administration and oral administration may be employed provided proper formulation is designed. In addition, it may be desirable to employ controlled release formulations using biodegradable films and matrices, or osmotic mini-pumps, or delivery systems based on dextran beads, alginate, or collagen. In summary, formulations are available for administering the antibodies of the invention and are well-known in the art and may be chosen from a variety of options. Typical dosage levels can be optimized using standard clinical techniques and will be dependent on the mode of administration and the condition of the patient.

The following examples are intended to illustrate but not to limit the invention. The examples hereinbelow employ, among others, a murine monoclonal antibody designated “266” which was originally prepared by immunization with a peptide composed of residues 13-28 of human Aβ peptide. The antibody was confirmed to immunoreact with this peptide. The preparation of this antibody is described in U.S. Pat. No. 5,766,846, incorporated herein by reference. As the examples here describe experiments conducted in murine systems, the use of murine monoclonal antibodies is satisfactory. However, in the treatment methods of the invention intended for human use, humanized forms of the antibodies of the present invention, or fragments thereof, are preferred.

### EXAMPLE 1

### Effect of Administration of Antibody 266 on Cognition in 24-month Old Transgenic, Hemizygous PDAPP Mice

Sixteen hemizygous transgenic mice (APP^{V717F}) were used. The mice were approximately 24 months old at the start of the study. All injections were intraperitoneal (i.p.). Half the mice received weekly injections of phosphate buffered saline (PBS, “Control”) and the other half received 355 micrograms of mouse antibody 266 dissolved in PBS. Injections were made over a period of seven weeks (42 days) for a total of six injections. Three days following the last injection, the behavior of the animals was assessed using an object recognition task, essentially as described in J.-C. Dodart, et al., Behavioral Neuroscience, 113 (5) 982-990 (1999). A recognition index (T_{B}×100)/(T_{B}-T_{A}) was calculated. Results are shown below in Table 1.

TABLE 1
Descriptive statistics for recognition index

| | | Recognition Index (minutes) | | |
|---|---|---|---|---|
| | | | Standard | Standard |
| | N | Mean | Deviation | Error |
| Control (PBS) | 8 | 71.2** | 8.80 | 3.11 |
| Antibody 266 | 8 | 54.35 | 7.43 | 2.62 |

**p = 0.0010

Administration of 355 micrograms of antibody 266 weekly to 24 month old, hemizygous, transgenic mice was associated with a significant change in behavior. Antibody treated transgenic mice had recognition indices which were similar to wildtype control animals [J.-C. Dodart, et al]. The difference in the recognition index was statistically significant at the 0.001 probability level. The increased recognition index is an indication that treatment with an antibody of the present invention will reverse the behavioral impairments that had been documented in this mouse model of Alzheimer's Disease. Therefore, the administration of the antibodies of the present invention, that bind Aβ more avidly than mouse 266, will treat diseases such as Alzheimer's disease and Down's syndrome and will halt the cognitive decline typically associated with disease progression.

The amyloid burden (% area covered by immunoreactive material after staining with anti-Aβ antibodies 3D6 or 21F12) was quantified in the cortex immediately overlying the hippocampus including areas of the cingulate and parietal cortex from the brains of the 24 month-old animals treated with mouse antibody 266 for seven weeks, as described above. The results are presented in the table below. The differences between the treatment groups are not statistically significant.

TABLE 2
Amyloid plaque burden in APP^{V717F +/-} mice following
treatment with mouse 266 anti-Aβ antibody

| | | Plaque Burden (%) | | | |
|---|---|---|---|---|---|
| | | Using 3D6 | | Using 21F12 | |
| | | | Standard | | Standard |
| | N | Mean | Error | Mean | Error |
| Control (PBS) | 7 | 44.3 | 5.93 | 0.77 | 0.14 |
| Antibody 266 | 8 | 38.0 | 2.96 | 0.93 | 0.11 |

For these very old animals, treatment with mouse antibody 266 did not result in a significantly different amyloid burden compared with the PBS-treated group, measured using either 3D6 or using 21F12. Furthermore, the Ap burden was substantially greater and significantly increased compared with the amyloid burden in younger animals (see below) who were not able to discriminate a novel object from a familiar one in the object recognition task. Most surprisingly, these results indicate that anti-Aβ antibodies of the present invention will most likely also be able to reverse cognitive deficits without the need to reduce amyloid burden per se.

### EXAMPLE 2

### Effect of Administration of Antibody 266 On Cognition in Young Transgenic, Hemizygous PDAPP Mice

Fifty-four (54) homozygous, transgenic mice (APP^{V717F}) were used. Twenty-three (23) mice were approximately two months old at the start of the study. The remaining mice were approximately four months old at the start of the study. The duration of treatment was five months. Thus, at study termination, the mice were either approximately seven (7) months old or approximately nine (9) months old.

All injections were intraperitoneal (i.p.). Each mouse in “PBS” control groups received a weekly injection of phosphate buffered saline (PBS; 200 μL). Each mouse in the “IgG” control groups received a weekly injection of IgG1κ isotype control (100 μg/mouse/week). Each mouse in the “High Dose” groups received a weekly injection of 355 microgram of antibody 266 dissolved in PBS (“HD”). Each mouse in the “Low Dose” group received a weekly injection of 71 microgram of antibody 266 dissolved in PBS (“LD”). Three days following the last injection, the behavior of the animals was assessed using an object recognition task, as described in Example 1 above, and a discrimination index was calculated as the difference between the time spent on a novel object and the time spent on a familiar object. Results are shown below in Table 3. The data are grouped by the age of the mice at the end of the study.

TABLE 3
Descriptive statistics for discrimination index

| | Discrimination Index (minutes) | | |
|---|---|---|---|
| | | Standard | Standard |
| | Mean | Deviation | Error |
| 7 months old | | | |
| PBS | 2.12 | 4.22 | 1.59 |
| IgG | 0.81 | 3.64 | 1.29 |
| HD | 10.04* | 6.52 | 2.30 |
| 9 months old | | | |
| PBS | 1.87 | 3.54 | 1.34 |
| IgG | 0.96 | 3.51 | 1.24 |
| LD | 10.75* | 6.44 | 2.28 |
| HD | 12.06*** | 7.82 | 2.76 |

*p < 0.05
***p < 0.0001

Taken together these data support the conclusion that administration of antibody 266 attenuates plaque deposition in 7-9 month old APP^{V717F} transgenic mice, as well as reverses the behavioral impairments previously characterized. Treatment of patients with an antibody of the present invention will inhibit or prevent cognitive decline typically associated with disease progression, and will reverse it.

### EXAMPLE 3

### Synthesis of Humanized Antibody 266 Cells and Antibodies.

Mouse myeloma cell line Sp2/0 was obtained from ATCC (Manassas, Va.) and maintained in DME medium containing 10% FBS (Cat # SH30071.03, HyClone, Logan, Utah) in a 37° C. CO₂ incubator. Mouse 266 hybridoma cells were first grown in RPMI-1640 medium containing 10% FBS (Hyclone), 10 mM HEPES, 2 mM glutamine, 0.1 mM non-essential amino acids, 1 mM sodium pyruvate, 25 μg/ml gentamicin, and then expanded in serum-free media (Hybridoma SFM, Cat # 12045-076, Life Technologies, Rockville, Md.) containing 2% low Ig FBS (Cat # 30151.03, HyClone) to a 2.5 liter volume in roller bottles. Mouse monoclonal antibody 266 (Mu266) was purified from the culture supernatant by affinity chromatography using a protein-G Sepharose column. Biotinylated Mu266 was prepared using EZ-Link Sulfo-NHS-LC-LC-Biotin (Cat # 21338ZZ, Pierce, Rockford, Ill.).

Cloning of variable region cDNAs. Total RNA was extracted from approximately 10⁷ hybridoma cells using TRIzol reagent (Life Technologies) and poly(A)⁺ RNA was isolated with the PolyATract mRNA Isolation System (Promega, Madison, Wis.) according to the suppliers' protocols. Double-stranded cDNA was synthesized using the SMART™TRACE cDNA Amplification Kit (Clontech, Palo Alto, Calif.) following the supplier's protocol. The variable region cDNAs for the light and heavy chains were amplified by polymerase chain reaction (PCR) using 3′ primers that anneal respectively to the mouse kappa and gamma chain constant regions, and a 5′ universal primer provided in the SMART™RACE cDNA Amplification Kit. For VL PCR, the 3′ primer has the sequence:

| | |
|---|---|
| 5′-TATAGAGCTCAAGCTTGGATGGTGGGAAGATGGATACAGTTGGTGC-3′ | [SEQ ID NO:13] |

with residues 17-46 hybridizing to the mouse Ck region. For VH PCR, the 3′ primers have the degenerate sequences:

| | |
|---|---|
| A G T | |
| 5′-TATAGAGCTCAAGCTTCCAGTGGATAGACCGATGGGGCTGTCGTTTTGGC-3′ | [SEQ ID NO:14] |
| T | |

with residues 17-50 hybridizing to mouse gamma chain CH 1. The VL and VH cDNAs were subcloned into pCR4Blunt-TOPO vector (Invitrogen, Carlsbad, Calif.) for sequence determination. DNA sequencing was carried out by PCR cycle sequencing reactions with fluorescent dideoxy chain terminators (Applied Biosystems, Foster City, Calif.) according to the manufacturer's instruction. The sequencing reactions were analyzed on a Model 377 DNA Sequencer (Applied Biosystems).

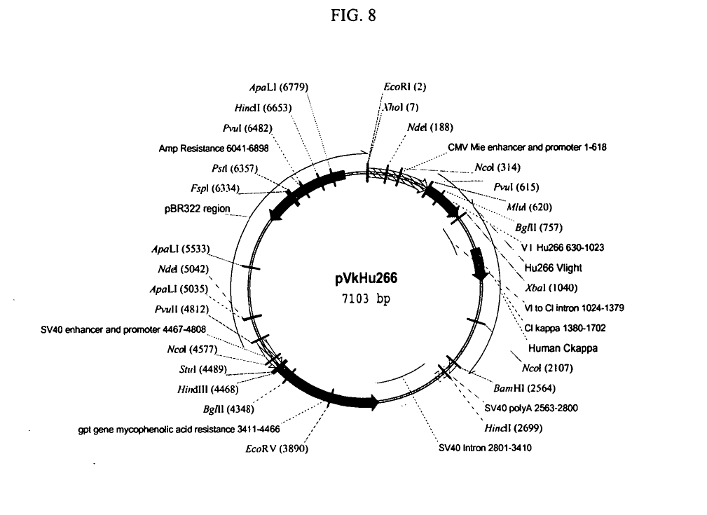

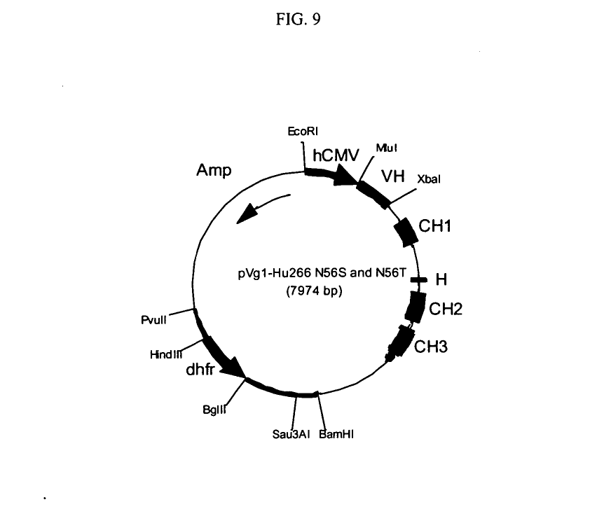

Construction of humanized 266 (Hu266) variable regions. The light and heavy chain variable region genes were constructed and amplified using eight overlapping synthetic oligonucleotides ranging in length from approximately 65 to 80 bases [He, X. Y., et al., J. Immunol. 160: 029-1035 (1998)]. The oligonucleotides were annealed pairwise and extended with the Kienow fragment of DNA polymerase I, yielding four double-stranded fragments. The resulting fragments were denatured, annealed pairwise, and extended with Klenow, yielding two fragments. These fragments were denatured, annealed pairwise, and extended once again, yielding a full-length gene. The resulting product was amplified by PCR using the Expand High Fidelity PCR System (Roche Molecular Biochemicals, Indianapolis, Ind.). The PCR-amplified fragments were gel-purified and cloned into pCR4Blunt-TOPO vector. After sequence confirmation, the VL and VH genes were digested with MIul and XbaI, gel-purified, and subcloned respectively into vectors for expression of light and heavy chains to make pVk-Hu266 (FIG. 8) and pVg1-Hu266 [Co, M. S., et al., J. Immunol. 148:1149-1154 (1992)]. The mature humanized 266 antibody expressed from these plasmids has the light chain of SEQ ID NO:11 and the heavy chain of SEQ ID NO:12.

Stable transfection. Stable transfection into mouse myeloma cell line Sp2/0 was accomplished by electroporation using a Gene Pulser apparatus (BioRad, Hercules, Calif.) at 360 V and 25 μF as described (Co et al., 1992). Before transfection, pVk-Hu266 and pVg1-Hu266 plasmid DNAs were linearized using FspI. Approximately 10⁷ Sp2/0 cells were transfected with 20 μg of pVk-Hu266 and 40 μpg of pVg1-Hu266. The transfected cells were suspended in DME medium containing 10% FBS and plated into several 96-well plates. After 48 hr, selection media (DME medium containing 10% FBS, HT media supplement, 0.3 mg/ml xanthine and 1 μg/ml mycophenolic acid) was applied. Approximately 10 days after the initiation of the selection, culture supernatants were assayed for antibody production by ELISA as shown below. High yielding clones were expanded in DME medium containing 10% FBS and further analyzed for antibody expression. Selected clones were then adapted to growth in Hybridoma SFM.

Measurement of antibody expression by ELISA. Wells of a 96-well ELISA plate (Nunc-Immuno plate, Cat # 439454, NalgeNunc, Naperville, Ill.) were coated with 100 μl of 1 μg/ml goat anti-human IgG, Fcγ fragment specific, polyclonal antibodies (Cat # 109-005-098, Jackson ImmunoResearch, West Grove, Pa.) in 0.2 M sodium carbonate-bicarbonate buffer (pH 9.4) overnight at 4° C. After washing with Washing Buffer (PBS containing 0.1% Tween 20), wells were blocked with 400 μl of Superblock Blocking Buffer (Cat # 37535, Pierce) for 30 min and then washed with Washing Buffer. Samples containing Hu266 were appropriately diluted in ELISA Buffer (PBS containing 1% BSA and 0.1% Tween 20) and applied to ELISA plates (100 μl per well). As a standard, humanized anti-CD33 IgG1 monoclonal antibody HuM195 (Co, et al., 1992, above) was used. The ELISA plate was incubated for 2 hr at room temperature and the wells were washed with Wash Buffer. Then, 100 μl of 1/1,000-diluted HRP-conjugated goat anti-human kappa polyclonal antibodies (Cat # 1050-05, Southern Biotechnology, Birmingham, Ala.) in ELISA Buffer was applied to each well. After incubating for I hr at room temperature and washing with Wash Buffer, 100 μl of ABTS substrate (Cat #s 507602 and 506502, Kirkegaard and Perry Laboratories, Gaithersburg, Md.) was added to each well. Color development was stopped by adding 100 μl of 2% oxalic acid per well. Absorbance was read at 415 nm using an OPTImax microplate reader (Molecular Devices, Menlo Park, Calif.).

Purification of Hu266. One of the high Hu266-expressing Sp2/0 stable transfectants (clone I D9) was adapted to growth in Hybridoma SFM and expanded to 2 liter in roller bottles. Spent culture supernatant was harvested when cell viability reached 10% or below and loaded onto a protein-A Sepharose column. The column was washed with PBS before the antibody was eluted with 0.1 M glycine-HCI (pH 2.5), 0.1 M NaCl. The eluted protein was dialyzed against 3 changes of 2 liter PBS and filtered through a 0.2 μm filter prior to storage at 4° C. Antibody concentration was determined by measuring absorbance at 280 nm (1 mg/ml=1.4 A₂₈₀). SDS-PAGE in Tris-glycine buffer was performed according to standard procedures on a 4-20% gradient gel (Cat # EC6025, Novex, San Diego, Calif.). Purified humanized 266 antibody is reduced and run on an SDS- PAGE gel. The whole antibody shows two bands of approximate molecular weights 25 kDa and 50 kDa. These results are consistent with the molecular weights of the light chain and heavy chain or heavy chain fragment calculated from their amino acid compositions.

### EXAMPLE 4

### In Vitro Binding Properties of Humanized 266 Antibody

The binding efficacy of humanized 266 antibody, synthesized and purified as described above, was compared with the mouse 266 antibody using biotinylated mouse 266 antibody in a comparative ELISA. Wells of a 96-well ELISA plate (Nunc-Immuno plate, Cat # 439454, NalgeNunc) were coated with 100 μI of β-amyloid peptide (1-42) conjugated to BSA in 0.2 M sodium carbonate/bicarbonate buffer (pH 9.4)(10 μg/mL) overnight at 4° C. The Aβ₁₋₄₂-BSA conjugate was prepared by dissolving 7.5 mg of Aβ₁₋₄₂-Cys₄₃ (C-terminal cysteine Aβ₁₋₄₂, AnaSpec) in 500 μL of dimethylsulfoxide, and then immediately adding 1,500 μL of distilled water. Two (2) milligrams of maleimide-activated bovine serum albumin (Pierce) was dissolved in 200 μL of distilled water. The two solutions were combined, thoroughly mixed, and allowed to stand at room temperature for two (2) hours. A gel chromatography column was used to separate unreacted peptide from Aβ₁₋₄₂-Cys-BSA conjugate.

After washing the wells with phosphate buffered saline (PBS) containing 0.1% Tween 20 (Washing Buffer) using an ELISA plate washer, the wells were blocked by adding 300 μL of SuperBlock reagent (Pierce) per well. After 30 minutes of blocking, the wells were washed Washing Buffer and excess liquid was removed.

A mixture of biotinylated Mu266 (0.3 μg/ml final concentration) and competitor antibody (Mu266 or Hu266; starting at 750 μg/ml final concentration and serial 3-fold dilutions) in ELISA Buffer were added in triplicate in a final volume of 100 PI per well. As a no-competitor control, 100 μl of 0.3 μg/ml biotinylated Mu266 was added. As a background control, 100 μl of ELISA Buffer was added. The ELISA plate was incubated at room temperature for 90 min. After washing the wells with Washing Buffer, 100 μl of 1 μg/ml HRP-conjugated streptavidin (Cat # 21124, Pierce) was added to each well. The plate was incubated at room temperature for 30 min and washed with Washing Buffer. For color development, 100 μl/well of ABTS Peroxidase Substrate (Kirkegaard & Perry Laboratories) was added. Color development was stopped by adding 100 μL/well of 2% oxalic acid. Absorbance was read at 415 nm. The absorbances were plotted against the log of the competitor concentration, curves were fit to the data points (using Prism) and the IC50 was determined for each antibody using methods well-known in the art.

The mean IC50 for mouse 266 was 4.7 μg/mL (three separate experiments, standard deviation=1.3 μg/mL) and for humanized 266 was 7.5 μg/mL (three separate experiments, standard deviation=1.1 μg/mL). A second set of three experiments were carried out, essentially as described above, and the mean IC50 for mouse 266 was determined to be 3.87 μg/mL (SD=0.12 μg/mL) and for human 266, the IC50 was determined to be 4.0 μg/mL (SD=0.5 μg/mL). On the basis of these results, we conclude that humanized 266 has binding properties that are very similar to those of the mouse antibody 266. Therefore, we expect that humanized 266 has very similar in vitro and in vivo activities compared with mouse 266 and will exhibit in humans the same effects demonstrated with mouse 266 in mice.

### EXAMPLE 5

### In Vitro Binding, Properties of Mouse Antibody 266 and Humanized Antibody 266

Antibody affinity (KD=Kd/Ka) was determined using a BlAcore biosensor 2000 and data analyzed with BlAevaluation (v. 3.1) software. A capture antibody (rabbit anti-mouse) was coupled via free amine groups to carboxyl groups on flow cell 2 of a biosensor chip (CM5) using N-ethyl-N-dimethylaminopropyl carbodiimide and N-hydroxysuccinimide (EDC/NHS). A non-specific rabbit IgG was coupled to flow cell 1 as a background control. Monoclonal antibodies were captured to yield 300 resonance units (RU). Amyloid-beta 1-40 or 1-42 (Biosource International, Inc.) was then flowed over the chip at decreasing-concentrations (1000 to 0.1 times KD). To regenerate the chip, bound anti-Aβ antibody was eluted from the chip using a wash with glycine-HCI (pH 2). A control injection containing no amyloid-beta served as a control for baseline subtraction. Sensorgrams demonstrating association and dissociation phases were analyzed to determine Kd and Ka. Using this method, the affinity of mouse antibody 266 for both Aβ₁₋₄₀ and for Aβ₁₋₄₂ was found to be 4 pM. The affinity of humanized 266 for Aβ₁₋₄₂ was found to be 4 pM.

### EXAMPLE 6

### Synthesis of Deglycosylated Humanized Antibody 266 Variants N56S and N56T

Site-directed mutagenesis. Site-directed mutagenesis was performed using the QuikChange XL Site-Directed Mutagenesis Kit (Cat # 200517, Stratagene, La Jolla, Calif.). To generate N56S and N56T variants in the VH CDR2 of Hu266, a pair of oligonucleotide primers containing the desired nucleotide substitution was designed according to the manufacturer's instructions. The primers were extended with PfuTurbo DNA polymerase using pVg1-Hu266 plasmid DNA as a template. The resulting product was treated with Dpn I endonuclease specific for methylated and hemimethylated DNA to digest the parental template. The resulting variant plasmids pVg1-Hu266 N56S and pVg1-Hu266 N56T were confirmed by sequencing.

Cell culture. Mouse myeloma cell line Sp2/0-Ag14 (referred to as Sp2/0 in this document; Cat # CRL-1581, ATCC, Manassas, Va.) was grown in DME medium containing 10% FBS (Cat # SH32661.03, Lot # AKE1 1827, HyClone, Logan, Utah) in a 37° C. CO₂ incubator. Selection for gpt expression was performed with DME medium containing 10% FBS, HT media supplement (Cat # H-0137, Sigma, St. Louis, Mo.), 0.3 mg/rnl xanthine (Cat # X-3627, Sigma) and 1 pg/ml mycophenolic acid (Cat # 11814-019, Life Technologies, Rockville, Md.).

Stable transfection. To establish cell lines producing variant Hu266, stable transfection into Sp2/0 was accomplished in essentially the same manner as described in Example 3. ELISA analysis occurred approximately 7 days after initiation of selection.

Measurement of antibody expression by ELISA. See Example 3 for ELISA details.

Sequencing of Hu266 light and variant heavy chain cDNA. Total RNA was isolated from approximately 2×10⁷ hybridoma cells using TRIzol reagent (Life Technologies). First-strand cDNA was synthesized using total RNA as a template and random hexadeoxynucleotides as primers. The reaction was performed with SuperScript II reverse transcriptase (Life Technologies) according to the supplier's protocol. DNA fragments containing the entire coding region of Hu266 light or variant heavy chain were amplified by PCR using 5′ and 3′ primers which bind to 5′ and 3′ non-coding regions, respectively. The amplified fragments were gel-purified and subjected to sequencing with appropriate primers.

Purification of variant Hu266. See Example 3 for purification details. The following differences are noted for clarity. For each variant Hu266, clone A4 was for Hu266 N56S and clone D2 for Hu266 N56T. The column was washed with PBS before the antibody was eluted with 0.1 M glycine-HCl (pH 2.8), 0.1 M NaCl. After neutralization with 1 M TrisHCl (pH 8), the eluted protein was dialyzed against 3 changes of 2 liters PBS and filtered through a 0.2 μm filter prior to storage at 4° C. SDS-PAGE in MES buffer was performed according to standard procedures on a 4-12% NuPAGE gel (Cat # NP032 1, Invitrogen). Gel staining was performed with the Colloidal Blue Staining Kit (Cat # LC6025, Invitrogen) according to the supplier's protocol.

### EXAMPLE 7

### Comparative Binding of Mouse 266, Humanized Antibody 266 Variants N56S and N56T

ELISA competition. Wells of 96-well ELISA plates (Nunc-Immuno plate, Cat # 439454, NalgeNunc) were coated with 100 μl of 3 μg/ml of BSA conjugated with □-amyloid peptide in 0.2 M sodium carbonate-bicarbonate buffer (pH 9.4) overnight at 4° C., washed with Wash Buffer, blocked with Superblock blocking buffer for 30 min at room temperature, and washed again with Wash Buffer. A mixture of biotinylated Mu266 (0.6 μg/ml final concentration) and competitor antibody (Mu266 or variant Hu266; typically starting at 750 μg/ml final concentration with serial 3-fold dilutions) in ELISA Buffer were added in triplicate in a final volume of 100 μl per well. As a no-competitor control, 100 μl of 0.6 μg/ml biotinylated Mu266 was used. As a background control, 100 μl of ELISA Buffer was used. ELISA plates were incubated at room temperature for 2 hr. After washing the wells with Washing Buffer, 100 μl of 10 μg/ml HRP-conjugated streptavidin (Cat # 21124, Pierce) was added to each well. ELISA plates were incubated at room temperature for 30 min and washed with Washing Buffer. For color development, 100 μl/well of ABTS substrate was added. Color development was stopped by adding 100 μl/well of 2% oxalic acid. Absorbance was read at 415 nm.

The affinities of Mu266, the original Hu266 (wild-type), Hu266 N56S and Hu266 N56T to β-amyloid peptide were compared by competition ELISA. Mu266, wild-type Hu266, Hu266 N56S and Hu266 N56T were competed with biotinylated Mu266 in a concentration-dependent manner. Hu266 N56S and Hu266 N56T showed affinities higher than Mu266 and the original Hu266. The IC₅₀ values of Mu266, Hu266 N56S and Hu266 N56T were obtained in three independent experiments for each variant. The values were calculated using the computer software Prism (GraphPad Software Inc., San Diego, Calif.) and are shown in Table 4. The relative binding affinities of Hu266 N56S and Hu266 N56T were on average 6.2-fold and 5.8-fold greater than that of Mu266, respectively. This represents a significant increase in affinity of the deglycosylated, variant humanized antibodies compared with the glycosylated (at position 56) mouse antibody.

TABLE 4
Summary of ELISA competition experiments
IC₅₀ (μg/ml)

| Competitor | Exp. I | Exp. II | Exp. III | Average | Std. Dev. |
|---|---|---|---|---|---|
| Mu266 | 3.8 | 4.5 | 6.1 | 4.8 | 0.96 |
| Hu266 N56S | 0.43 | 0.92 | 1.0 | 0.78 | 0.25 |
| Difference | 8.8 fold | 4.9 fold | 6.1 fold | 6.2 fold | |

IC₅₀ (μg/ml)

| Competitor | Exp. I | Exp. II | Exp. III | Average | Std. Dev. |
|---|---|---|---|---|---|
| Mu266 | 4.3 | 6.4 | 6.4 | 5.7 | 0.99 |
| Hu266 N56T | 0.68 | 1.2 | 1.1 | 0.99 | 0.23 |
| Difference | 6.3 fold | 5.3 fold | 5.8 fold | 5.8 fold | |

### EXAMPLE 8

### Affinity of Humanized Antibody 266 Variant N56S and N56T

Antibody affinity (KD=Kd/Ka) was determined using a BIAcore biosensor 2000 and data analyzed with BIAevaluation (v. 3.1) software in essentially the same manner as described in Example 5. ELISA experiments were conducted in essentially the same manner as described in Example 7.

The data below show that the deglycosylated humanized antibody variants (N56S, N56T) have significantly better affinity than the glycosylated form (h266). While interanalysis variations exist, these differences have no significant affect on the relative affinity improvement demonstrated for these deglycosylated variants over the glycosylated form.

Affinity-BIAcore

| | KD |
|---|---|
| | (pM) |
| N56S | 2.5 |
| H266 | 7.2 |
| N56T | 1.87 |
| h266 | 3.47 |

Competitive Binding (IC50, μg/mL)-ELISA

| | Mean | S.D. | N |
|---|---|---|---|
| N56S | 1.9 | 0.2 | 3 |
| Hu 266 | 7.2 | 1.9 | 3 |
| Mu 266 | 7.6 | 1.4 | 3 |
| N56T | 3.1 | 0.9 | 3 |
| Hu 266 | 13.0 | 2.6 | 3 |
| Mu 266 | 14.0 | 2.6 | 3 |
| N56S | 0.43 | — | 1 |
| Mu 266 | 3.80 | — | 1 |
| N56S | 1.0 | — | 1 |
| N56T | 1.0 | — | 1 |
| Hu 266 | 5.0 | — | 1 |
| Mu 266 | 7.1 | — | 1 |

### EXAMPLE 9

### Determination of Glycosylation at Position 56 of the Heavy Chain of Humanized Antibody 266

For each of two lots of humanized 266 that had been expressed and purified essentially as described above, a sample was prepared containing approximately 100 μg antibody. Each sample was reduced by adding 50 mg urea, 5 μL of 50 mg/mL DTT and 10 μL of 3 M tris buffer, pH 8.0 and incubating at 37° C. for 30 min. The protein was alkylated by adding 20 tL of 50 mg/mL iodoacetamide solution and incubating at room temperature in the dark for 30 min. The solution was desalted on 1 mL spin column packed with P-6 resin. The desalting columns were washed and eluted with 0.025 M NH₄HCO₃ buffer. About 250 μL of protein fraction was collected for each sample. Each protein fraction was mixed with 2 to 3 μL of 1 mg/mL trypsin solution, and then the mixture was incubated at 37° C. for about 2.5 hours. The remaining trypsin activity was quenched by heating the solution at 100° C. for 3 minutes. For desialylated samples, 10 μL of tryptic digests of each sample was mixed with 7 μL of 0.15% formic acid in water and 2 μL of neuraminidase (a. u.) solution (1 unit/mL). The mixture was incubated at 37° C. for 1 to 3 hours before HPLC/MS analysis. For de-N-glycosylated sample, 10 μL of tryptic digest was treated with 1 μL of N-glycosidase F at 37° C. for 3 hours.

All the solutions were directly analyzed by capillary HPLC/MS with the following conditions: HPLC was an HP100; Column: Zorbax C8, 2.1×150 mm or Vydac C18, 0.3×150 mm; Temperature: ambient; Flow rate: 200 μL/min for Zorbax, 5-10 μL/min for C18; Injection volume: 10 μL after 1:1 dilution or original solution; HPLC solvents: A-0.15% formic acid in H₂O, B-0.12% formic acid in ACN; Gradient (time, %B): (0,2), (40,50), (43,90), (45,90), (46,2), (50,2); mass spectrometry: API 150EX MASS SPEC 03, step 0.333, DP 25 V, ISV 5000 V, and FP 250 V.

In both lots analyzed, two peaks were found to contain glycopeptides. After de-N-glycosylation, new peptide masses, 1189.6 and 1672.5, in one of the peaks (eluted around 13 minutes) were found. These two masses match the heavy chain 288-296 and 284-296 (expected masses after de-N-glycosylation: 1190.2 and 1672.8). In the other peak (eluted about 26 minutes) a new peptide mass, 2369.4, was found after de-N-glycosylation. This mass matches the heavy chain peptide 44-65. Hence, the potential glycosylation sites, Asn 56 and 292 of the heavy chain were glycosylated. No clear peaks were found on the reconstructed ion chromatograms of peptides 288-296 and 44-65 from HPLC/MS analysis of tryptic digests. The results indicated that the Asn 56 site was fully glycosylated for both lots of humanized 266 antibody.

1-28. (cancelled)

29. an antibody, or fragment thereof, comprising a light chain and a heavy chain, wherein the light chain comprises the three light chain complementarity determining regions (CDRs) from mouse monoclonal antibody 266 (SEQ ID NO: 1-3), and wherein the heavy chain comprises heavy chain CDR1 and CDR3 from mouse monoclonal antibody 266 (SEQ ID NO:4 and 6, respectively), and a heavy chain CDR2 having the sequence given by SEQ ID NO:5:
Gln Iie Asn Ser Val Gly Xaa Xaa Xaa Tyr Tyr Pro Asp Thr Val Lys Gly (SEQ ID NO:5)
wherein,
Xaa at position 7 of SEQ ID NO:5 is any amino acid, provided that if Xaa at position 8 is neither Asp nor Pro and Xaa at position 9 is Ser or Thr, then Xaa at position 7 is not Asn;
Xaa at position 8 of SEQ ID NO:5 is any amino acid, provided that if Xaa at position 7 is Asn and Xaa at position 9 is Ser or Thr, then Xaa at position 8 is Asp or Pro; and
Xaa at position 9 of SEQ ID NO:5 is any amino acid, provided that if Xaa at position 7 is Asn and Xaa at position 8 is neither Asp nor Pro, then Xaa at position 9 is neither Ser nor Thr.

30. The antibody or fragment of claim 29, wherein:
Xaa at position 7 of SEQ ID NO:5 is selected from the group consisting of Ala, Gly, His, Asn, Gln, Ser, and Thr, provided that if Xaa at position 9 is Ser or Thr, then Xaa at position 7 is not Asn;
Xaa at position 8 of SEQ ID NO:5 is selected from the group consisting of Ala, Gly, His, Asn, Gln, Ser, and Thr; and
Xaa at position 9 of SEQ ID NO:5 is selected from the group consisting of Ala, Gly, His, Asn, Gln, Ser, and Thr, provided that if Xaa at position 7 is Asn, then Xaa at position 9 is neither Ser nor Thr.

31. The antibody or fragment of claim 30, wherein Xaa at position 7 of SEQ ID NO:5 is Ala, Gly, His, Gln, Ser, or Thr, or His, Xaa at position 8 is Ser, and Xaa at position 9 is Thr.

32. The antibody or fragment of claim 31, wherein Xaa at position 7 of SEQ ID NO:5 is Ser or Thr, Xaa at position 8 is Ser, and Xaa at position 9 is Thr.

33. The antibody or fragment of claim 29, wherein Xaa at position 8 of SEQ ID NO:5 is Ser and Xaa at position 9 is Thr.

34. The antibody or fragment of claim 29, wherein Xaa at position 7 of SEQ ID NO:5 is Asn and Xaa at position 8 is Ser.

35. The antibody or fragment of claim 29 having a light chain variable region of the sequence given by SEQ ID NO:7 and a heavy chain variable region given by SEQ ID NO:8.

36. The antibody or fragment of claim 35, wherein in the heavy chain:
Xaa at position 56 is selected from the group consisting of Ala, Gly, His, Asn, Gln, Ser, and Thr, provided that if Xaa at position 58 is Ser or Thr, then Xaa at position 56 is not Asn;
Xaa at position 57 is selected from the group consisting of Ala, Gly, His, Asn, Gln, Ser, and Thr; and
Xaa at position 58 is selected from the group consisting of Ala, Gly, His, Asn, Gln, Ser, and Thr, provided that if Xaa at position 56 is Asn, then Xaa at position is neither Ser nor Thr.

37. The antibody or fragment of claim 36, wherein in the heavy chain Xaa at position 56 is Ala, Gly, His, Gln, Ser, or Thr, Xaa at position 57 is Ser, and Xaa at position 58 is Thr.

38. The antibody or fragment of claim 37, wherein in the heavy chain Xaa at position 56 is Ser or Thr, Xaa at position 57 is Ser, and Xaa at position 58 is Thr.

39. The antibody or fragment of claim 35, wherein in the heavy chain Xaa at position 57 is Ser and Xaa at position 58 is Thr.

40. The antibody or fragment of claim 35, wherein in the heavy chain Xaa at position 56 is Asn and Xaa at position 57 is Ser.

41. The antibody or fragment thereof of claim 35 having a light chain variable region of the sequence given by SEQ ID NO:9 and a heavy chain variable region given by SEQ ID NO:10.

42. The antibody or fragment thereof of claim 41 having a light chain of the sequence given by SEQ ID NO:11 and a heavy chain of the sequence given by SEQ ID NO:12.

43. An antibody fragment obtainable by enzymatic cleavage of the antibody of claim 29.

44. An antibody fragment of claim 29 which is a Fab or F(ab′)2 fragment.

45. The antibody or fragment of claim 29 that is an IgG₁ immunoglobulin isotype.

46. The antibody or fragment of claim 29, wherein the antibody or fragment thereof is produced in a host cell selected from the group consisting of a myeloma cell, a chinese hamster ovary cell, a syrian hamster ovary cell, and a human embryonic kidney cell.

47. A polynucleotide comprising a sequence coding for the light chain or the heavy chain of the antibody or fragment of claim 29, or a fragment thereof.

48. A polynucleotide which when expressed in a suitable host cell, yields a light chain or a heavy chain of the antibody of claim 29, or a fragment thereof.

49. An expression vector for expressing the antibody of claim 29 comprising the polynucleotide of claim 48.

50. A cell transfected with the expression vector of claim 49.

51. A cell transfected with two expression vectors of claim 49, wherein a first vector comprises the polynucleotide coding for the light chain and a second vector comprises the polynucleotide coding for the heavy chain.

52. A cell that is capable of expressing a humanized antibody of claim 29.

53. A pharmaceutical composition that comprises the humanized antibody or fragment of claim 29, and a pharmaceutically acceptable excipient.


SEQUENCE LISTING (17 sequences)

SEQ ID NO: 1 - protein, 16 aa, Mus sp.
  MISC_FEATURE (1)..(16): LIGHT CHAIN CDR1
RSSQSLIYSDGNAYLH

SEQ ID NO: 2 - protein, 7 aa, Mus sp.
  MISC_FEATURE: LIGHT CHAIN CDR2
KVSNRFS

SEQ ID NO: 3 - protein, 9 aa, Mus sp.
  MISC_FEATURE (1)..(9): LIGHT CHAIN CDR3
SQSTHVPWT

SEQ ID NO: 4 - protein, 5 aa, Mus sp.
  MISC_FEATURE (1)..(5): HEAVY CHAIN CDR1
RYSMS

SEQ ID NO: 5 - protein, 17 aa, Mouse variant
  MISC_FEATURE (1)..(17): HEAVY CHAIN CDR2
QINSVGXXXYYPDTVKG

SEQ ID NO: 6 - protein, 3 aa, Mus sp.
  MISC_FEATURE (1)..(3): HEAVY CHAIN CDR3
GDY

SEQ ID NO: 7 - protein, 113 aa, Artificial Sequence
  Humanized antibody
DXVMTQXPLSLPVXXGQPASISCRSSQSLXYSDGNAYLHWFLQKPGQSPXLLIYKVSNRFSGVPDRFSGSGSGTDFTLKISRVEAEDXGVYYCSQSTHVPWTFGXGTXXEIKR

SEQ ID NO: 8 - protein, 112 aa, Artificial Sequence
  Humanized antibody
XVQLVEXGGGLVQPGGSLRLSCAASGFTFSRYSMSWVRQAPGKGLXLVAQINSVGXXXYYPDXVKGRFTISRDNXXNTLYLQMNSLRAXDTAVYYCASGDYWGQGTXVTVSS

SEQ ID NO: 9 - protein, 113 aa, Artificial Sequence
  Humanized antibody
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKR

SEQ ID NO: 10 - protein, 112 aa, Artificial Sequence
  Humanized antibody
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSS

SEQ ID NO: 11 - protein, 219 aa, Artificial Sequence
  Humanized antibody
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 442 aa, Artificial Sequence
  Humanized antibody
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 17 aa, Artificial Sequence
  Humanized antibody
QINSVGXXXYYPDTVKG

SEQ ID NO: 14 - protein, 17 aa, Artificial Sequence
  Humanized antibody
QINSVGXXXYYPDTVKG

SEQ ID NO: 15 - protein, 17 aa, Artificial Sequence
  Humanized antibody
QINSVGXXXYYPDTVKG

SEQ ID NO: 16 - protein, 17 aa, Artificial Sequence
  Humanized antibody
QINSVGXXXYYPDTVKG

SEQ ID NO: 17 - protein, 17 aa, Artificial Sequence
  Humanized antibody
QINSVGXXXYYPDTVKG